(19) United States
(12) Patent Application Publication
(10) Pub. No.: US 2004/0121336 A1
(43) Pub. Date: Jun. 24, 2004

(54) Method for generating multiple samples containing a predetermined amount of nucleic acid

(76) Inventors: I Lawrence Greenfield, San Mateo, CA (US); Douglas A. Bost, San Mateo, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/325,588

(22) Filed: Dec. 20, 2002

(51) Int. Cl. C12Q 1/68

(52) U.S. Cl. 435/6

(57) ABSTRACT

A method is provided for binding a predetermined amount of a nucleic acid. The method involves contacting one or more sample solutions comprising a nucleic acid with a multiplicity of solid substrate binding units, where the solid substrate binding units bind the nucleic acid. Each binding unit has a predetermined binding capacity for the nucleic acid. An apparatus for binding one or more predetermined amounts of a nucleic acid is also provided.

### BACKGROUND OF THE INVENTION

Nucleic acid manipulation and analysis is at the heart of modern biological, medical, and agricultural research. Researchers often need to isolate nucleic acid from a biological sample for assay, manipulation, or analysis. A researcher may want to isolate total nucleic acid, or selectively isolate DNA or RNA.

Selective binding of nucleic acid to solid substrates is one way to purify nucleic acids from contaminants in a biological sample.

The quantity of a solid substrate contacted with a single sample of nucleic acid also generally has a variable or unknown binding capacity for the nucleic acid. Where particles of a solid substrate are used, the size, shape, and surface area of the particles, and the surface density of the component of the solid substrate responsible for binding the nucleic acid are all generally variable, which leads to individual solid substrate particles, or a given volume or mass of particles, having variable and uncertain binding capacity. It is often not known which factors will affect the binding capacity of the solid substrate and in what way they will affect it. Further, there are few solid substrates that selectively bind DNA and not RNA where the starting material is a mixture of both. Even with those substances that do selectively bind DNA, the selectivity often is not good and the factors responsible for the selective binding are generally not well established.

One type of commonly used nucleic acid binding kit uses particles of a silica-based material, such as diatomaceous earth, to bind the nucleic acid. A slurry of particles is mixed with the sample to bind the nucleic acid. The particles, now holding bound nucleic acid, are separated by centrifugation and washed to remove unbound materials. With this type of kit, it is difficult to isolate a reproducible and consistent quantity of nucleic acid for analysis, particularly if the quantity of nucleic acid desired for an individual assay is small. Individual particles of diatomaceous earth are very small and of variable sizes, shapes, and surface areas. Thus, it is not practical to physically isolate a single particle or a specific number of particles and elute the nucleic acid for analysis. Even if this were possible, the variability in the size and shapes of the particles means that the amount of DNA bound by a single particle or small number of particles would be variable.

Even if a larger quantity of nucleic acid is desired for a single assay, it is difficult to use diatomaceous earth to provide a consistent quantity. The amount of diatomaceous earth needed to purify nucleic acid for a single assay is ordinarily small, so it is difficult and inconvenient for a researcher to measure a consistent weight of diatomaceous earth to be added to each sample to purify nucleic acid. Even if aliquots of diatomaceous earth of a consistent weight could be obtained, the variability in particle size and other characteristics between aliquots would mean the aliquots might not bind a consistent quantity of nucleic acid.

There are several consequences to delivering too much DNA to downstream analytical processes. For example, when running GENESCAN analytical processes, too much DNA can result in off-scale data resulting in inaccurate multi-component correction of the data. Incomplete spectral separation can then result in pull-up peaks that lead to incorrect genotyping of a forensic sample. Amplification of too much DNA can also cause incomplete Clarke reaction resulting in the presence of additional peaks. Again, the presence of additional peaks can complicate the genotyping of samples and, in rare cases, can result in the confusion with mixed or rare genotypes. Amplification of too much DNA complicates DNA quantification.

A need exists for methods for isolating a consistent predetermined quantity of nucleic acid from a single sample or separate samples for analysis. This need is particularly great when the predetermined quantity desired is small and it is desired from separate, non-identical samples. There is also a need for a method where multiple containers can hold the same predetermined amount of nucleic acid. A need also exists for methods to control the binding capacity of a solid substrate for nucleic acid. There is also a need for methods to selectively extract DNA or RNA from a mixture containing both types of nucleic acid using a solid substrate. In the absence of selectivity, it is difficult to obtain a predetermined quantity of the intended species (DNA or RNA).

### SUMMARY OF SOME EMBODIMENTS OF THE INVENTION

In order to deliver a predetermined amount of either RNA or DNA or total nucleic acid, both the type of nucleic acid isolated and the amount of nucleic acid recovered needs to be controlled. Some embodiments of the present invention provide methods for selectively isolating either RNA or DNA, and methods and compositions for isolating a known amount or concentration of the selected nucleic acid species. Some embodiments of the present invention also provide methods and compositions for nonselectively isolating a known amount of total nucleic acid from a mixture of RNA and DNA, where the nucleic acid isolated is a mixture of RNA and DNA.

Some embodiments of the present invention provide a method for binding a predetermined amount of a nucleic acid. The method involves contacting one or more sample solutions of the nucleic acid with a multiplicity of solid substrate binding units. The solid substrate binding units bind the nucleic acid. Each binding unit has a predetermined binding capacity for the nucleic acid.

For example, the solid substrate binding units can be silica particles of a defined shape, size, and surface area. It has been discovered that the binding capacity of silica particles for both DNA and RNA varies with the pH, the identity of anions and cations in the binding buffer, and with salt concentration. Under certain conditions, the silica particles selectively bind DNA and do not significantly bind RNA. Thus, by controlling these factors, an investigator can selectively isolate a known quantity of substantially pure DNA or a known quantity of substantially pure RNA from a mixture of both.

The present invention also provides an apparatus for binding one or more predetermined amounts of a nucleic acid. The apparatus contains a multiplicity of solid substrate binding units. The solid substrate binding units bind the nucleic acid under appropriate conditions. Each binding unit has a substantially identical binding capacity for the nucleic acid as compared to the other binding units, or has a binding capacity that is a predetermined ratio of the binding capacity of another unit. The binding units are operably linked to form an array, or are arranged so as to make simultaneous contact with a single sample solution of the nucleic acid.

### BRIEF DESCRIPTION OF THE DRAWINGS

FIG. 1 shows the amount of DNA bound to various solid substrates in the presence of different salts at pH 8.

FIG. 2 shows the amount of DNA bound to various solid substrates in the presence of different anions at pH 8.

FIG. 3 shows the amount of RNA bound to various solid substrates in the presence of different salts at pH 8.

FIG. 4 shows the effect of salt on DNA selectivity based on the data shown in FIGS. 1 and 3 (i.e., the ratio of DNA recovered in FIG. 1 to RNA recovered in FIG. 3).

FIGS. 5(a)-(k) show the effects of pH and salt on DNA binding to various solid substrates.

FIG. 6 shows the amount of RNA bound to two solid substrates as a function of pH and salt.

FIG. 7 shows a comparison of the effect of NaCl and NaI on the DNA selectivity (i.e., the ratio of DNA/RNA recovery).

FIGS. 8(a)-(c) show the DNA selectivity of various solid substrates at pH 8 or 10 in NaI.

FIG. 9 shows a comparison of the amount of DNA bound versus the amount of RNA bound to various solid substrates at pH 8 or 10 in NaI.

FIG. 10 shows the quantity of DNA or RNA recovered from silica particles as a function of input quantity.

FIG. 11 shows the quantity of protein (bovine serum albumin) recovered from silica in NaI as a function of binding pH.

FIGS. 12(a)-(f) show the effect of cation and anion on DNA and RNA binding to silica solid substrates at pH 10.

FIG. 13 shows sequential selective binding and sequential selective elution schemes for separate isolation of DNA and RNA from a mixture of both.

FIG. 14 shows the relationship of DNA binding capacity to solid substrate surface area of silica particles.

FIG. 15 shows binding capacity per unit mass of solid substrate particle as compared to particle surface area using silica particles.

FIG. 16 shows the effects of sodium iodide concentration on binding capacity for DNA of a silica solid substrate.

### DETAILED DESCRIPTION

Definitions

“Solid substrate” includes rigid and flexible solids. Examples of solid substrates include gels, fibers, microspheres, spheres, cubes, particles of other shapes, channels, microchannels, capillaries, walls of containers, and filters. The solid substrate stably binds a nucleic acid. By “stably binds” it is meant that under defined binding conditions the equilibrium substantially favors binding over release of the nucleic acid, and if the solid substrate containing a selected bound nucleic acid is washed with buffer lacking nucleic acid under these defined binding conditions, substantially all the nucleic acid remains bound. In particular embodiments the binding is reversible. By “reversible” it is meant that under defined elution conditions the bound nucleic acid is predominantly released from the solid substrate and can be recovered in solution. In particular embodiments, at least 80%, at least 90%, or at least 95% of the bound nucleic acid is released under the defined elution conditions.

The binding interactions of the solid substrate with the nucleic acid may be hydrogen bonds, ionic interactions, hydrophobic interactions, or even covalent interactions, or a combination thereof. The solid substrates can consist entirely of one material that binds a nucleic acid, or can contain more than one component, at least one of which is exposed on the surface of the solid substrate and binds a nucleic acid.

“Binding component” refers to a component of a solid substrate that binds nucleic acids. Examples of binding components include various types of silica, including glass and diatomaceous earth, anion exchange groups such as diethylaminoethane (DEAE), cation exchange groups such as carboxylates (see, e.g., U.S. Pat. Nos. 5,705,628 and 5,898,071), immobilized dyes that bind nucleic acids (e.g., methidium, ethidium, and ethidium homodimer), and hydrophobic interaction groups. Thus, examples of solid substrates include silica particles, magnetic beads coated with silica, and resins coated with anion or cation exchange groups, hydrophobic interaction groups, or dyes.

“Binding unit” refers to a physically or functionally distinct quantity of the solid substrate that is capable of conveniently contacting a single sample solution of a nucleic acid without the sample solution contacting other binding units. Examples include a single well of a multiwell plate, a single microchannel, or a single particle of silica. A “binding unit” can also be a physically or functionally distinct quantity of the solid substrate that is part of an array, where the binding units in the array can simultaneously contact a single sample solution of a nucleic acid, where after the contacting the individual binding units are conveniently separable from each other at predetermined separation points. For instance, the binding units could be polyhedrons connected in an array by linkers, where the polyhedrons can be conveniently separated from the linkers at predetermined points.

“Contacting conditions” include conditions under which a solid substrate contacts (e.g., binds to) a nucleic acid. These conditions include pH, presence or absence of buffer, identity and concentration of buffer when present, the concentration and type of salts in a sample solution, and the presence or absence of other components in the sample solution, such as proteins, carbohydrates, cells, and tissues. In addition, contacting conditions include the temperature and time the nucleic acid sample solution is in contact with the solid substrate. Contacting conditions can also include the relative concentrations and types of different nucleic acids present in the sample solution. For example, the sample solution may contain DNA or RNA or both, and within the categories of DNA and RNA may contain particular types of DNA or RNA, e.g., supercoiled DNA, linear or relaxed DNA, DNA complexed to histones, naked DNA, ribosomal RNA or messenger RNA.

“Washing conditions” include conditions under which unbound or undesired components are removed from a solid substrate containing a bound target nucleic acid. The unbound or undesired component removed by washing can be separately analyzed. Washing conditions include pH, presence or absence of buffer, identity and concentration of buffer when present, the concentration and type of salts in a sample solution, and the presence or absence of other components in the sample solution. In addition, washing conditions include the temperature and time the washing solution is in contact with the solid substrate containing the bound target nucleic acid. Contacting conditions can be adjusted to remove nontarget biological components bound (either specifically or nonspecifically) to the solid substrate. For example, the wash solution may be adjusted to remove bound protein from the solid substrate, while retaining bound RNA and DNA. Alternatively, the wash solution may, for example, be adjusted to remove protein and RNA while retaining bound DNA on the solid substrate.

“Elution conditions” include conditions under which the nucleic acid of interest is released from the solid substrate containing the bound target nucleic acid. These conditions include pH, presence or absence of buffer, identity and concentration of buffer when present, the concentration and type of salts in a sample solution, and the presence or absence of other components in the sample solution. In addition, elution conditions include the temperature and time the elution solution is in contact with the solid substrate containing the bound target nucleic acid.

“Substantially identical contacting conditions” refers to the conditions in a single sample solution contacted simultaneously with a multiplicity of units, as well as the conditions of contacting separate units with separate sample solutions where the sample solutions are prepared identically except for the inherent unavoidable variation between independently prepared samples.

“Binding capacity” refers to the amount of nucleic acid bound to a solid substrate binding unit when the amount bound no longer increases substantially with contacting the unit with increasing quantities of the nucleic acid under the same contacting conditions. Binding capacity is a characteristic of the identity of the solid substrate, the surface area of the solid substrate binding unit, and the contacting conditions.

The term “predetermined binding capacity” as used herein refers to a binding capacity that is known for a particular type of nucleic acid under particular contacting conditions. Hence, the term “binding a predetermined amount of a nucleic acid” refers to an amount that is predetermined if the contacting conditions and type of nucleic acid in the sample are known.

The term “substantially identical binding capacity” refers to binding capacity that is sufficiently consistent for the user's purposes and is as consistent as can reasonably be achieved with different units made to be as consistent as is reasonably feasible. The binding capacity of different units contacted with a sample under substantially identical contacting conditions preferably varies by less than about ±30%. In other embodiments, it varies by less than about ±20%, less than about ±10%, or less than about ±5%. The binding capacity may vary with the type of nucleic acid bound. For instance, it may vary depending on whether the nucleic acid bound is RNA, DNA, or a mixture, and on whether DNA is supercoiled, relaxed, or single stranded.

“Sample solution comprising a nucleic acid” refers to a liquid in which the nucleic acid is substantially in solution when the nucleic acid contacts the solid substrate binding unit. Other components in the solution may also be in solution, or may be in suspension or may be present in the sample solution without being in solution or suspension.

“Silica-based material” or “silica-based solid substrate” refers to a material having a significant amount of silicon dioxide or composed predominantly of silicon dioxide. This includes, for instance, materials referred to by various manufacturers as silica, silicon dioxide, diatomaceous earth, glass, and quartz.

“Anion exchange group” refers to cationic groups used to bind anionic groups. These include, for instance, tertiary and quaternary amines.

“Cation exchange group” refers to anionic groups used to bind cationic groups. These include, for instance, carboxylates and sulfates.

“Hydrophobic interaction group” refers to groups that interact with materials by hydrophobic interactions. These include, for instance, octyl and phenyl groups.

“Cation” includes compounds that can be positively charged at an appropriate set of buffer conditions. These compounds include positively charged protonated compounds in equilibrium with the corresponding uncharged, unprotonated compounds, such as ammonium-containing compounds in equilibrium with ammonia-containing compounds. Other examples include Li⁺, Na⁺, K⁺, Cs⁺, Mg⁺⁺, and Ca⁺⁺.

“Anion” includes compounds that can be negatively charged at an appropriate set of buffer conditions. These compounds include negatively charged unprotonated compounds in equilibrium with the corresponding uncharged protonated compounds, such as carboxylate-containing compounds in equilibrium with carboxylic acid-containing compounds. Other examples include F⁻, Cl⁻, Br⁻, I⁻, S²⁻, and Se²⁻.

“Preferentially binds DNA over RNA” means that the solid substrate binds more DNA than RNA under the conditions of the contacting. The solid substrate may bind just slightly more DNA than RNA, about twice as much, about three times as much, or about four times as much or greater DNA as RNA under the conditions of the contacting.

“Selectively binds DNA” means that the ratio of the amount of DNA bound to the solid substrate as compared to the amount of RNA bound to the solid substrate under the conditions of the contacting, washing, or eluting is at least about 5:1. In other specific embodiments, the ratio is at least about 10:1, at least about 15:1, at least about 20:1, or about 5:1 to about 30:1.

“Nucleic acid” and “oligonucleotide” refer to both DNA and RNA polynucleotides, as well as polynucleotides that include modified nucleotides. “Modified nucleotides” include, for example, dideoxyribonucletides and synthetic nucleotides having modified base moieties or modified sugar moieties, e.g., as described in Scheit, Nucleotide Analogs (John Wiley, New York 1980) and Uhlman and Peyman, Chemical Reviews 90:543-584 (1990). Such analogs include synthetic nucleotides designed to enhance binding properties, reduce degeneracy, and increase specificity. Nucleic acids and oligonucleotides will ordinarily involve nucleosides linked by phosphodiester linkages. In some cases, the phosphodiester linkage may be modified, as for instance, with ³⁵S-labeled phosphorothionate groups. Oligonucleotides can contain from 4 to several hundred nucleotides. Typically, they will contain from 10 to 100 nucleotides. In specific embodiments, the oligonucleotides can contain 4 to 100 nucleotides, 10 to 80 nucleotides, 10 to 60 nucleotides, or 10 to 40 nucleotides. Nucleic acids bound to the solid substrate binding units can be any length and any type, including oligonucleotides, genomic DNA, plasmids, viral nucleic acid, single-stranded and double-stranded nucleic acid, etc.

“Container” refers to a vessel capable of holding a substance, such as volume of fluid or a quantity of a solid substrate.

“Simultaneous contact” refers to more than one binding unit in contact with a single sample solution at the same time. For instance, two physically separate spherical binding units, such as spherical silica particles, are in simultaneous contact with a sample solution when they are both in a single container that is holding the sample solution. Thus, an apparatus containing a multiplicity of binding units where the binding units are arranged so as to make simultaneous contact with a single sample solution can be a collection of physically unconnected binding units, such as a collection of silica particles.

The indefinite articles “a” and “an,” and the definite article “the” are used, as is common in patent documents, to mean one or more unless the context clearly indicates otherwise. For example, “a nucleic acid” refers to one or more of such compounds.

“Bulky anion” refers to an anion with a molecular radius at least as large as bromide. Examples of bulky anions include bromide, iodide, perchlorate, thiocyanate, picrate, tannate, tungstate, molybdate, trichloroacetate, sulfosalicylate, tribromoacetate, and nitrate.

DESCRIPTION

Method for Isolating a Predetermined Quantity of Nucleic Acid

One embodiment of the present invention provides a method for binding a predetermined amount of a nucleic acid to a solid substrate. The method involves contacting a nucleic acid sample with a solid substrate binding unit. The binding unit is a physically or functionally distinct entity that can be conveniently contacted individually with the nucleic acid sample solution, without the sample solution contacting other binding units. The binding unit could be, for instance, a sphere, a collection of spheres, a polyhedron, a collection of polyhedrons, or a well of a multiwell plate. Each binding unit contains a solid substrate that binds the nucleic acid.

The nucleic acid can be DNA, RNA, or a mixture of the two. The nucleic acid can also be a structurally distinct type of nucleic acid, such as single-stranded DNA or RNA, double-stranded DNA or RNA, or supercoiled, relaxed, or linearized DNA. The binding units may be substantially identical to each other and have a substantially identical binding capacity for the nucleic acid of interest under substantially identical conditions. By varying the sample conditions in which the binding units contact the nucleic acid, however, the binding capacity of the units can be altered in a controlled way. Thus, different predetermined amounts can be bound by contacting different binding units with one or more samples under different conditions.

Another embodiment of the present invention provides a method of releasing a predetermined amount of a nucleic acid by eluting a predetermined amount of the nucleic acid bound to a solid substrate.

Another embodiment of the present invention provides a method for delivering a predetermined amount of a nucleic acid to a multiplicity of reaction vessels. The method involves contacting a nucleic acid sample with a multiplicity of solid substrate binding units that bind the nucleic acid. The individual binding units containing bound nucleic acid can be separated and delivered to separate reaction vessels for analysis of the nucleic acid or for use of the nucleic acid in a reaction.

Method to Selectively Extract DNA or RNA from a Mixture Containing Both Types of Nucleic Acid Using a Solid Substrate

One embodiment of the invention provides methods to selectively extract DNA or RNA from a mixture containing both types of nucleic acid by using differential binding to a solid substrate. The inventors have discovered conditions where DNA substantially binds to certain solid substrates, while RNA does not substantially bind. In general, silica-based solid substrates selectively bind DNA and do not bind substantial amounts of RNA or protein at a high pH in the presence of high concentrations of bulky anions. The term “bulky anion” refers to an anion with a molecular radius at least as large as bromide. Examples of bulky anions include bromide, iodide, perchlorate, thiocyanate, picrate, tannate, tungstate, molybdate, trichloroacetate, sulfosalicylate, tribromoacetate, and nitrate. Iodide gives the best selectivity of the anions.

The choice of cation also affects DNA vs. RNA selectivity. Among cations, sodium gives the highest selectivity for DNA over RNA.

“Washing conditions” include conditions under which components other than the nucleic acid of interest are removed from a solid substrate containing a bound target nucleic acid. These conditions include pH, presence or absence of buffer, identity and concentration of buffer when present, the concentration and type of salts in a sample solution, and the presence or absence of other components in the sample solution. In addition, washing conditions include the temperature and time the washing solution is in contact with the solid substrate containing the bound target nucleic acid. Washing conditions can be adjusted to remove nontarget biological components bound (either specifically or nonspecifically) to the solid substrate. For example, the wash solution may be adjusted to remove bound protein from the solid substrate, while retaining bound RNA and DNA. Alternatively, the wash solution may be adjusted to remove protein and RNA while retaining bound DNA on the solid substrate.

“Elution conditions” include conditions under which the targeted nucleic acid is released from the solid substrate containing the bound target nucleic acid. These conditions include pH, presence or absence of buffer, identity and concentration of buffer when present, the concentration and type of salts in a sample solution, and the presence or absence of other components in the sample solution. In addition, washing conditions include the temperature and time the washing solution is in contact with the solid substrate containing the bound target nucleic acid.

One embodiment of the invention provides methods to selectively extract a known quantity of DNA from a mixture containing both types of nucleic acids using selective binding to a solid substrate binding unit. The sample containing a mixture of both types of nucleic acids is contacted with a silica-based solid substrate at high pH (i.e., at least about 8) in the presence of high concentrations of bulky anions. Under these conditions, DNA, but not RNA, will selectively bind to the silica-based solid substrate. Since RNA will not compete for binding to the solid substrate, this approach allows for controlled binding of DNA to the solid substrate binding unit. Following removal of the unbound RNA by subsequent washing, the predetermined quantity of DNA can be recovered by elution, or the solid substrate binding unit containing a predetermined quantity of DNA can be used directly in a subsequent analysis.

An alternate embodiment of the invention provides methods to selectively extract a known quantity of RNA from a mixture containing both types of nucleic acids using selective binding to a solid substrate. DNA can be removed from a sample containing a mixture of both types of nucleic acids by first contacting the sample with a silica-based solid substrate at high pH in the presence of high concentrations of bulky anions. Under these conditions, DNA, but not RNA, will selectively bind to the solid substrate. The solid substrate containing the DNA can then be removed. The sample solution can then be adjusted to conditions that will permit RNA to bind to the solid substrate, such as by lowering the pH. The sample containing the RNA is then contacted with a silica-based solid substrate binding unit under conditions compatible with RNA binding. Following subsequent washing, the predetermined quantity of RNA can then be recovered by elution or the solid substrate binding unit containing the predetermined quantity of RNA can be used directly in a subsequent analysis.

In another embodiment of the invention, both DNA and RNA can be selectively extracted from a mixture containing both types of nucleic acid using selective binding to a solid substrate. The sample containing a mixture of both types of nucleic acids is contacted with a first amount of silica-based solid substrate binding units at high pH in the presence of high concentrations of bulky anions. Under these conditions, DNA, but not RNA, will selectively bind to the solid substrate binding units. The solid substrate binding units containing the bound DNA can then be separated from the remainder of the sample. The remainder of the sample is altered to permit binding of RNA to the solid substrate and a second amount of solid substrate binding units is then added. Once the RNA has bound to the solid substrate binding units, the supernatant depleted in nucleic acid is then removed. The two amounts of solid substrate binding units are then washed and the bound DNA and RNA is then separately recovered by elution. Alternatively, the bound DNA and RNA can be analyzed by using the solid substrate binding units with bound nucleic acid directly in an analysis.

In a fourth embodiment of the invention, both DNA and RNA can be selectively extracted from a mixture containing both types of nucleic acid using selective elution from a solid substrate. The sample containing a mixture of both types of nucleic acids is contacted with a first aliquot of silica-based solid substrate binding units under conditions that will permit the binding of both DNA and RNA. The solid substrate binding units containing bound DNA and RNA are subsequently washed under conditions that will elute the RNA but retain the DNA. In general, silica-based solid substrates selectively bind DNA and do not bind substantial amounts of RNA or protein at a high pH in the presence of high concentrations of bulky anions. The solid substrate containing the bound DNA is separated from the wash solution containing the eluted RNA. The conditions of the wash solution are adjusted to permit subsequent binding of RNA to a solid substrate. For example, the pH of the wash solution can be reduced to below pH 8. Subsequently, a second aliquot of solid substrate binding units is added. Once the RNA has bound to the solid substrate binding units, the supernatant depleted of nucleic acid is removed. The two aliquots of solid substrate binding units can then be washed and the bound DNA and RNA then separately recovered by elution. Alternatively, the DNA and RNA can be analyzed by using the solid substrate binding units with bound nucleic acid directly in an analysis assay.

Methods to Control the Binding Capacity of a Solid Substrate for Nucleic Acid

One embodiment of the present invention provides methods to control the binding capacity of a solid substrate for nucleic acids. This embodiment is in part based on the inventors' determination of the binding capacity of certain solid substrates for total nucleic acid and for DNA or RNA under various conditions. The inventors have discovered that the binding capacity of the solid substrates varies with the characteristics of the solid substrate and the conditions of the binding solution and have identified several factors that affect the binding capacity of the solid substrates. The identity and concentration of cations in the sample solution can affect the binding capacity. Lithium and sodium both allow high capacity of silica-based substrates for DNA. Lithium allows a higher capacity of silica-based substrates for RNA than do sodium or potassium. The pH of the sample solution during binding also affects the binding capacity. Lower pHs allow a higher capacity of silica-based substrates for RNA. The choice of anion also affects the binding capacity. For instance, the binding capacity of silica-based substrates for DNA increases in the order of Cl⁻<Br⁻<I⁻. Thiocyanate and perchlorate also give higher binding capacities of silica-based substrates for DNA than does chloride. The choice of anion also affects the binding capacity for RNA. Salt concentration also affects the binding capacity of solid substrates for nucleic acids. High salt concentrations generally increase the binding capacity of silica-based solid substrates for both RNA and DNA. Thus, the invention provides methods to control the binding capacity of a solid substrate for total nucleic acid or a particular nucleic acid.

One element of the present invention is a solid substrate binding unit, where the binding unit is capable of capturing a predetermined amount of nucleic acid target. The predetermined amount of nucleic acid chosen typically is an appropriate amount for a single assay or manipulation, such as PCR amplification, nucleic acid sequencing, restriction analysis, cloning, or detection by hybridization. Each binding unit may have a substantially identical binding capacity for the nucleic acid target. Alternatively, each binding unit may have a binding capacity that is a predetermined ratio of the binding capacity of another binding unit under identical contacting conditions. In one embodiment, one or more binding units are placed in a single container and a sample containing the target nucleic acid sample is added to the container, allowing capture of the target nucleic acid to generate binding units with a predetermined quantity of bound target nucleic acid. The binding units are subsequently placed, for instance, in one or more individual detection wells in a plate, or archived for storage. In one embodiment of the invention, an assay is performed on the nucleic acid while it is bound to the solid substrate binding unit. In another embodiment, the nucleic acid is eluted from the solid substrate binding unit before being used in an assay.

In another embodiment, the solid substrate is the wall of an individual well. Thus, the solid substrate acts both as a container and as the agent that binds the nucleic acid.

One example of a nucleic acid-binding solid substrate is silica. Melzak et al. (Melzak, K. A., Sherwood, C. S., Turner, R. F. B., and Haynes, C. A. (1996), Driving Forces For DNA Adsorption To Silica In Perchlorate Solutions, Journal of Colloid and Interface Sciences 181: 635-644) describes studies to determine the binding forces responsible for binding of DNA to silica surfaces. The authors used a microcrystalline silica particle (BDH Chemicals) that had a surface area of 5.6 (±0.2) m²/g. In 6 M sodium perchlorate solution at a pH from 3 to 5, the particles bound 800 mg supercoiled DNA/m².

Each human cell contains the equivalent of 6.6 pg of genomic DNA, so 1000 cells contain 6.6 ng of genomic DNA. Based on the findings of Melzak et al., a particle with a surface area of 8.25×10⁻⁶ m² has sufficient binding capacity to bind 6.6 ng of DNA. Such a particle, if spherical, has a radius of 8.1×10⁻⁴ m (810 μm) and a volume of 2.2 μL.

Solid substrates with higher binding capacities will permit particles of smaller volumes. Examples of solid substrates that bind DNA include, for example, aluminum oxide, zirconium oxide, titanium oxides, zeolites, aluminum hydroxide, diatomaceous earth, glass, boron silicates, aluminum silicates, ion exchange resins (e.g., DEAE-, QAE-, or PEI-derivatized solid supports), cellulose based paper (e.g., FTA paper), and dye-derivatized resins (e.g., methidium, crystal violet, ethidium bromide, Lucifer yellow, and acridine).

The amount of nucleic acid bound to the solid substrate binding unit is determined by the binding capacity of the binding units under particular contacting conditions (provided the amount of nucleic acid in the sample solution is greater than the binding capacity of the binding units). Different solid substrates are capable of binding different quantities of nucleic acid. Given the same surface density of the binding component of the solid substrate, the binding capacity of a binding unit is generally proportional to the surface area of the solid substrate binding unit under a particular contacting condition. In order for the binding capacity to be uniform and predictable, it is preferable that the solid substrate not be porous.

The binding capacity of the binding unit may be controlled by the sample solution contacting conditions. The binding capacity may be controlled by the pH of the sample solution during the contacting. The binding capacity may be controlled by the type of one or more cations in the sample during the contacting, and by the type of one or more anions in the sample during the contacting. Exemplary cations that alter the binding capacity of the binding units under the contacting conditions include lithium, sodium, potassium, rubidium, tetramethyl ammonium, tetraethyl ammonium, tetrapropyl ammonium, tetrabutyl ammonium, and a combination thereof. Exemplary anions that alter the binding capacity of the binding units under the contacting conditions include chloride, fluoride, phosphate, sulfate, sulfide, bromide, iodide, perchlorate, thiocyanate, picrate, tannate, tungstate, molybdate, acetate, trichloroacetate, sulfosalicylate, tribromoacetate, thiocyanate, trichloroacetate, nitrate, and combinations thereof.

The amount of nucleic acid bound to the binding units can also be controlled by the concentration of one or more salts (i.e., combinations of cations and anions) in the sample during the contacting. For instance, the amount can be controlled by the concentration of sodium iodide in the sample.

In a particular embodiment, after the binding units are contacted with the nucleic acid sample solution, the binding units are washed to remove components other than the bound nucleic acid.

In a specific embodiment, the nucleic acid sample solution is contacted with each of a multiplicity of binding units under substantially identical conditions. For instance, multiple binding units are placed in simultaneous contact with a single nucleic acid sample solution.

In another specific embodiment, different binding units are placed in contact with aliquots of a nucleic acid sample under different conditions.

The method for binding a predetermined amount of a nucleic acid can further involve, after the contacting step, the step of washing the binding units to remove components other than the nucleic acid.

In one specific embodiment of the invention, the solid substrate preferentially binds DNA over RNA.

In one specific embodiment of the invention, the solid substrate selectively binds DNA. In a particular embodiment where the solid substrate selectively binds DNA, the sample solution during the contacting with the binding units has a pH of at least about 8, and contains a salt (or a combination of salts) where the anion has a molecular radius at least as large as bromide. The salt may be present at a concentration of at least about 1 M. The solid substrate may be a silica-based material or may contain a silica-based material.

In another specific embodiment of the invention, the target nucleic acid is RNA. In order to separate the RNA from any DNA present in the sample, the RNA and DNA may first be bound to the binding units during the contacting, followed by selectively eluting the RNA by contacting the binding units with elution buffer. The contacting with the elution buffer may be under conditions wherein the solid substrate selectively binds DNA. In one embodiment of that invention, the elution buffer has a pH of at least about 8 and contains a salt (or a combination of salts) at a concentration of at least about 1 M, where the anion of the salt has a molecular radius at least as large as bromide. In a specific embodiment of this example, the solid substrate is a silica-based material or contains a silica-based material.

In one embodiment of the method for binding a predetermined amount of a nucleic acid, the sample is purified to remove one or more undesired components before the contacting step. For instance, protein may be removed from the sample.

In another embodiment, the nucleic acid desired is RNA and the undesired component to be removed is DNA. In one embodiment of that method, the DNA is removed from the sample by contacting the sample with a solid substrate under conditions where the solid substrate selectively binds DNA. For instance, the DNA is removed from the sample by contacting the sample with a solid substrate wherein the sample during the contacting with the solid substrate has a pH of at least about 8 and contains a salt (or a combination of salts) at a concentration of at least about 1 M, where the anion of the salt has a molecular radius at least as large as bromide. In a specific embodiment of this example, the solid substrate is or contains a silica-based material. In other embodiments, the concentration of the salt in the sample is at least about 2 M or at least about 3 M. In other embodiments, the sample has a pH of at least about 9, about 8 to about 12, about 9 to about 12, or about 9 to about 11, or any pH level in between.

In particular embodiments of conditions where the solid substrate binding units substantially bind DNA and substantially do not bind RNA, the sample or elution buffer has a pH of at least about 8. In other embodiments, the sample, wash, or elution buffer has a pH of at least about 9, about 8 to about 12, about 9 to about 12, or about 9 to about 11, or any pH level in between any of these values. In particular embodiments of these conditions, the sample, wash, or elution buffer contains a salt (or a combination of salts) at a concentration of at least about 1 M, where the anion of the salt has a molecular radius at least as large as bromide. In other embodiments, the concentration of the salt is at least about 2 M or at least about 3 M.

In particular embodiments, the anion is a monovalent anion. In other embodiments, the anion is divalent or multivalent. Particular anions suitable for use in these conditions for selective binding of DNA are bromide, iodide, perchlorate, thiocyanate, picrate, tannate, tungstate, molybdate, trichloroacetate, sulfosalicylate, tribromoacetate, and nitrate.

In particular embodiments of the selective binding conditions, the cation is sodium. In one particular embodiment, the salt is sodium iodide.

In a particular embodiment of the method for binding a predetermined amount of a nucleic acid, the nucleic acid is a mixture of DNA and RNA. In other particular embodiments, the nucleic acid is DNA or RNA. In other particular embodiments, the nucleic acid has a particular sequence, or is a particular type of nucleic acid, such as mRNA.

In a particular embodiment of the method for binding a predetermined amount of a nucleic acid, the binding units are magnetic. The individual magnetic units can be delivered to individual assay vessels by methods described in WO 97/40385 (Seul, Michael) or U.S. Pat. No. 5,396,136.

Non-magnetic particles can be delivered to individual assay vessels by a method such as an automated reagent delivery system (see U.S. Pat. No. 6,432,712 and U.S. published patent application No. 20020015666).

In particular embodiments of the invention, the solid substrate has as a binding component an anion exchange group or a silica-based material. In particular embodiments, the silica-based material is silica, diatomaceous earth, glass, quartz, or silica gel.

In other particular embodiments of the invention, the solid substrate contains as a binding component zirconium, titanium dioxide, a dye, a hydrophobic interaction group, an oligonucleotide, or an antibody. In other particular embodiments, the solid substrate contains as a binding component a metal oxide, such as aluminum hydroxide.

Dyes suitable for use in the solid substrate for binding nucleic acids include, for instance, methidium, crystal violet, ethidium bromide, Lucifer yellow, and acridine.

Nucleic acid binding components can be attached to other components of the solid substrate by any method or linkage known in the art. See, e.g., Advanced Organic Chemistry, Part B: Reactions and Synthesis, Second Edition, Cary and Sundberg (1983); Advanced Organic Chemistry, Reactions, Mechanisms, and Structure, Second Edition, March (1977); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999).

Oligonucleotide-linked magnetic particles are disclosed, for instance, in U.S. Pat. No. 5,512,439. Dye-based capture methods are disclosed, for instance, in U.S. Pat. Nos. 4,335,226; 5,453,186; and 4,921,805. Metal oxide supports for DNA capture are disclosed in U.S. Pat. No. 5,438,129 and WO 92/18514. Magnetic silica particles are disclosed, for instance, in U.S. Pat. Nos. 3,900,415; 4,360,441; and 4,554,088; and in WO 98/12717 and WO 98/31840. Other magnetic solid substrates are disclosed, e.g., in WO 91/12079; EP 0 515 484 B1; and in U.S. Pat. Nos. 5,898,071; and 5,523,231.

In a particular embodiment, the nucleic-acid-binding component of the solid substrate is not an oligonucleotide. In a particular embodiment, the solid substrate does not comprise an immobilized oligonucleotide.

In one embodiment of the invention, the binding units are containers, such as the wells of a multiwell plate. In other particular embodiments of the invention, the binding units are spheres, polyhedrons, microchannels, membranes, or filters.

The present invention also provides an apparatus for binding one or more predetermined amounts of a nucleic acid. The apparatus is made up of a multiplicity of solid substrate binding units. The solid substrate binding units are capable of binding the nucleic acid under appropriate conditions. Each binding unit may have a substantially identical binding capacity for the nucleic acid under substantially identical conditions. Alternatively, each binding unit may have a binding capacity that is a predetermined ratio of the binding capacity of another binding unit under substantially identical conditions. The binding units may be operably linked to form an array or may be arranged so as to make simultaneous contact with a single sample solution of the nucleic acid. For instance, if arranged so as to make simultaneous contact with a single sample solution of the nucleic acid, the binding units may be physically separate particles that are placed together in the sample solution of the nucleic acid.

An exemplary apparatus is a multiwell plate, where each well is a solid substrate binding unit. Another exemplary apparatus is a collection of spherical or polyhedron particles. In this case, each particle is a binding unit. The particles may be unconnected or may be physically linked in an array. If linked together, the linkages may be broken by physical or chemical means. Another example of an apparatus is a collection of microchannels, where the interior walls of the microchannels are composed of or coated with a material that binds the selected nucleic acid. The microchannels may be unconnected or may be physically linked in an array.

In a particular embodiment, the binding units are non-spherical.

In particular embodiments of the apparatus, the solid substrate contains a silica-based material, an anion exchange group, zirconium, titanium dioxide, a dye, a hydrophobic interaction group, an oligonucleotide, or an antibody.

In a particular embodiment of the apparatus, the binding units are containers, such as wells of a multiwell plate. In other particular embodiments, the binding units are polyhedrons, microchannels, membranes, or filters.

In order that the invention may be more readily understood, reference is made to the following examples, which are intended to illustrate the invention, but not limit the scope thereof.

### EXAMPLES

The following terms, abbreviations, and sources apply to the materials discussed throughout the Examples.

Solid substrates were obtained from the following sources: ORGANON TEKNIKA silica (Organon Teknika, Product Number 82951, Lot 00030302), diatomaceous earth (Sigma, Product Number D-3877, Lot 128H3702), EMPORE FILTER AID 400 (3M, Product Number 56221-746, Lot 990020), silica gel (J. T. Baker, Product Number 3405-01, Lot N36338), SIGMA silica (Sigma, Product Number S-5631, Lot 58H0154), Binding Matrix (BIO-101, Product Number 6540-408, Lot Number 6540-408-0B13), GLASSMILK (BIO 101, Product Number 2072-204, Lot Number 2072-204-8A17), DAVISIL Grade 643 Silica Gel (Spectrum, Product Number Sil 66, Lot NE 0387), and Uniform Silica Microspheres (Bangs Laboratories, Inc. Catalog Code SS05N, Inv # L0002188).

Abbreviations or names of the following reagents and sources for them are as follows: guanidine hydrochloride (Sigma, Lot 38H5432), guanidine thiocyanate (Sigma, Product Number G-9277), sodium iodide (Aldrich Chemical Company, Product Number 38,311-2, Lot Number 07004TS), sodium perchlorate (Aldrich Chemical Company, Product Number 41,024-1, Lot KU 06910HU), sodium bromide (Aldrich, Product Number 31050-6, Lot 11805KR), sodium chloride (Aldrich Chemical Company, Product Number 33,251-4, Lot Number 16524CS), Tris (TRIZMA base, Tris[Hydroxymethyl]aminomethane, Sigma, Product Number T-6791, Lot Number 126115738), MES (2-[N-Morpholino]ethanesulfonic acid, Sigma, product number M5287, lot number 58H5411), AMP (2-amino-2-methyl-1-propanol, Sigma, Product Number 221), Hepes (n-[2-hydroxyethyl]piperazine-N′-[2-ethane sulfonic acid], Sigma, product number H-4034, lot number 19H54101), ethanol (Ethyl alcohol, absolute, Aldrich, catalog number E702-3), HCl (Sigma, Product Number H7020, Lot Number 97H3562), sodium hydroxide (Sigma, Product Number S-8045, Lot Number 127HO531 and 69H1264), ammonium bifluoride (ammonium hydrogen fluoride, Aldrich Product Number 22,482-0), nitric acid (Aldrich, Product number 22571-1, lot number 00261 Al), and ammonium hydroxide (Aldrich product number 22,122-8, lot number 02308KR).

Nucleic acids and tissue samples were obtained from the following sources: calf thymus genomic DNA (deoxyribonucleic acid, type 1, highly polymerized from calf thymus, Sigma, Product Number D-1501, Lot 87H7840); rat liver total RNA (Biochain Institute, lot numbers A304057, A305062, or A306073); and whole human blood (Blood Centers of the Pacific).

The spectrophotometry was performed with a Hewlett-Packard Model 8453 Spectrophotometer.

Gel electrophoresis of nucleic acid samples in Examples 12 to 14 was carried out using SEAKEM agarose (Teknova); 1×TBE (89 mM Tris, 89 mM Boric Acid, 2 mM EDTA; Teknova, catalog number 0278-1L, lot number 17F801); and 0.5 μg/ml ethidium bromide buffer (Bio-Rad). Molecular weight markers used in electrophoresis were an AMPLISIZE DNA molecular weight standard (Bio-Rad), a High Molecular Weight DNA Marker (Gibco BRL), and an RNA ladder (Gibco BRL).

In the Examples below, a quantity of nucleic acid is contacted with a solid substrate and bound to the solid substrate. The bound nucleic acid is eluted from the substrate and the amount in solution after elution is quantified. The amount of nucleic acid measured in solution after binding and elution is referred to as the “recovered” nucleic acid. The amount of nucleic acid recovered in the Examples closely matches the amount bound to the solid substrates, because elution under the conditions used is generally quantitative. Thus, the amount recovered can be used as a proxy for the amount bound. The Examples refer to “binding” where this is the concept being addressed by the experiment, but it should be understood that it is the quantity of recovered DNA that is being measured, since it is difficult to directly measure the quantity of nucleic acid bound to the solid substrate.

### Example 1

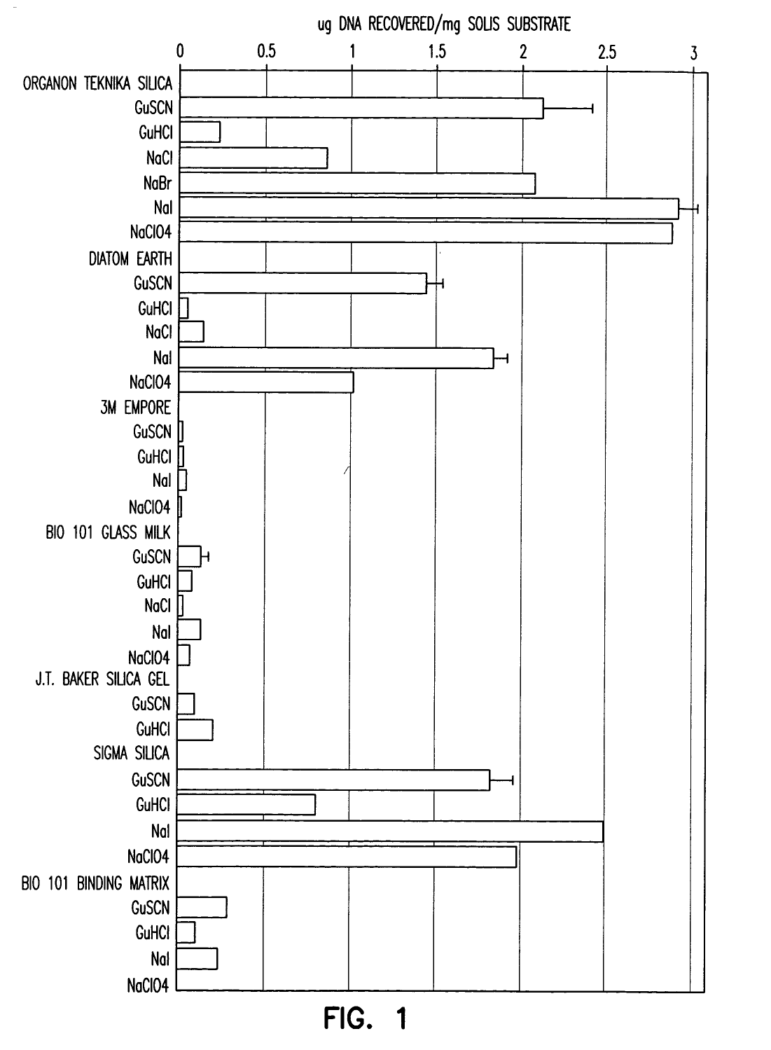

Several solid substrates were tested in the following example. These solid substrates were silica and glass matrices from a variety of suppliers. They were evaluated for their ability to bind genomic DNA using different salts at pH 8. A number of screening experiments were initially performed evaluating the effect of various solid substrates and salt compositions. FIG. 1 represents a consolidation of at least six experiments.

ORGANON TEKNIKA silica, diatomaceous earth, EMPORE FILTER AID 400, J. T. BAKER silica gel, SIGMA silica, BIO101Binding Matrix, and GLASSMILK were used for the following example. Prior to use, all particles (except the silica from Organon Teknika) were prepared as follows: the particles were washed once with 4-8 volumes of 1 N HCl, twice with 4-8 volumes of water; once with 4-8 volumes of 1 N NaOH, twice with 4-8 volumes of water, once with 4-8 volumes of ethanol, and four times with 4-8 volumes of water. As used herein, one “volume” of water or ethanol refers to an amount of water or ethanol equal in mass to the mass of the particles being washed. The Binding Matrix and GLASSMILK from BIO-101 were washed 4 times with at least 4 volumes of water before being treated with HCl, NaOH, and ethanol (as described above). The ORGANON TEKNIKA silica particles were used as supplied. Diatomaceous earth, EMPORE FILTER AID 400, J. T. BAKER silica gel, and SIGMA silica were stored as a 200 mg/ml (20%) slurry in water. The BIO101 Binding Matrix particles were stored as a 580 mg/ml slurry in water and the GLASSMILK particles were stored as a 373 mg/ml slurry in water.

Calf thymus DNA was used as the source of the genomic DNA. Sheared genomic DNA used in the following examples was prepared as follows. The DNA was resuspended at approximately 10 mg/ml in water. The DNA was then sheared by passing the material four times through a 20 gauge 1½ inch needle, three times through a 21 gauge 1½ inch needle and 22 gauge 1½ inch needle, and once through a 26 gauge 1½ inch needle.

Each solid substrate and buffer combination shown in FIG. 1 was assayed once using the following procedure. Sheared calf thymus DNA (30 μg of DNA, 50 μl of a 0.5 mg/ml concentration) was added to 1.5 ml microcentrifuge tubes containing 0.45 ml of one of the following buffers: (1) 50 mM Tris-HCl, pH 8, 4.75 M guanidine thiocyanate; (2) 50 mM Tris HCl, pH 8; 4.75 M guanidine hydrochloride; (3) 50 mM Tris HCl, pH 8; 4.75 M sodium chloride; (4) 50 mM Tris-HCl, pH 8, 4.75 M sodium bromide; (5) 50 mM Tris-HCl, pH 8, 4.75 M Sodium iodide; or (6) 50 mM Tris-HCl, pH 8, 4.75 M sodium perchlorate. The nucleic acid was incubated for up to 10 minutes at ambient temperature in the buffered solution, with occasional mixing. Solid substrates (10 mg ORGANON TEKNIKA silica, 10 mg diatomaceous earth, 10 or 100 mg 3M EMPORE, 19 or 190 mg GLASSMILK, 10 mg J. T. BAKER silica gel, 10 mg SIGMA silica, and 10 or 20 mg BIO101 Binding Matrix) were added separately to each of the buffered nucleic acid solutions. The solutions were incubated for 10 minutes with the solid substrates at ambient temperature with occasional mixing to allow the nucleic acid to bind to the solid substrate. The particles were then centrifuged (15,800×g, 1 minute) and washed twice with 0.5 ml of the binding buffer that had been used for the binding incubation. Subsequently, the particles were washed three to four times with 0.5 ml of 70% ethanol.

Following the last ethanol wash, the particles were allowed to air dry at ambient temperature or at 56° C. for 5-10 minutes. The bound nucleic acid was first eluted from the solid substrate with 0.25 ml of 10 mM Tris, pH 9, for 5 minutes at 56° C. with constant mixing and the eluted nucleic acid was collected. Residual nucleic acid bound to the particles was subsequently eluted with 0.25 ml of 0.1 N NaOH at 56° C. for 5 minutes with constant mixing, and the eluted nucleic acid was collected. The amount of nucleic acid was quantified by spectrophotometry. See Sambrook et al., Molecular Cloning: A Laboratory Manual (2d ed., Cold Spring Harbor Laboratory Press, Plainview, N.Y.). The results are shown in FIG. 1. The amount of DNA recovered is expressed per mg of solid substrate to normalize for the different amounts of solid substrate initially added. When multiple determinations were measured in separate experiments, the average and standard deviation are shown.

At a binding pH of 8, the effect of salt composition of the binding solution on DNA recovery varied depending on the solid substrate used. For a particular solid substrate, the recovery of DNA varied up to 43-fold, depending on the choice of salt in the binding solution. Anions that gave the highest binding capacities for DNA for most of the solid substrates in this work were the bulky anions thiocyanate, bromide, iodide, and perchlorate. In general, recovery of DNA was poor with salts containing the less bulky chloride anion. A comparison of the effect of GuHCl and GuSCN on DNA binding demonstrated greater DNA recovery in the presence of the larger thiocyanate anion.

### Example 2

The effect of two anions on DNA binding to several solid substrates was examined more carefully. The solid substrates used in the present example were prepared as described in Example 1. Sheared genomic DNA from calf thymus was prepared as described in Example 1.

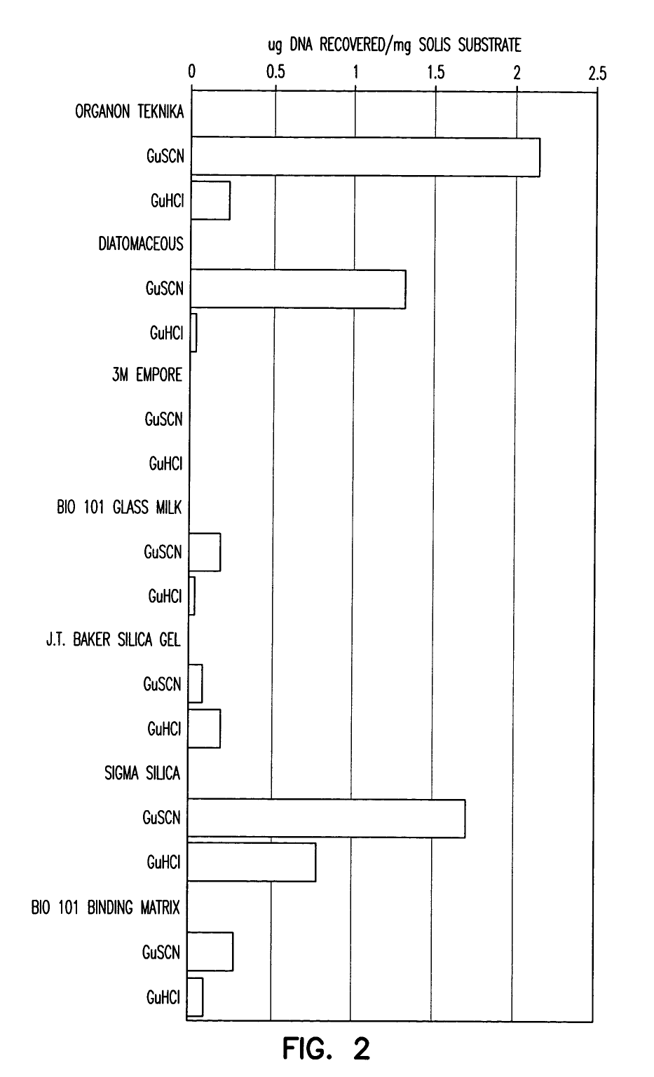

Each solid substrate and buffer combination was assayed once as follows. Sheared calf thymus DNA (25 μg of DNA, 50 μl of a 0.5 mg/ml concentration) was added to a 1.5 ml microcentrifuge tube containing 0.45 ml of either (1) 50 mM Tris-HCl, pH 8 and 4.75 M guanidine thiocyanate; or (2) 50 mM Tris-HCl, pH 8 and 4.75 M guanidine hydrochloride. Nucleic acid was incubated up to 10 minutes at ambient temperature in the buffered solution, with occasional mixing. The seven solid substrates (10 mg ORGANON TEKNIKA silica, 10 mg diatomaceous earth, 10 mg 3M EMPORE, 18.6 mg GLASSMILK, 10 mg J. T. BAKER silica gel, 10 mg SIGMA silica, 28.8 mg BIO101 Binding Matrix) were added separately to each of the two buffered nucleic acid solutions so that in total there were 14 containers. The solutions were incubated for 10 minutes with the solid substrates at ambient temperature with occasional mixing. Washing of the particles, elution of the nucleic acid, and quantitation of the eluted nucleic acid was performed as in Example 1. The results are shown in FIG. 2. In general, recovery of DNA after binding in the presence of thiocyanate was superior to recovery after binding in the presence of chloride ions. The quantity of DNA recovered varied also with the solid substrate. Low quantities of DNA were recovered from 3M EMPORE, GLASSMILK, and J. T. BAKER silica gel.

### Example 3

The recovery of RNA using silica and glass solid substrates from a variety of suppliers was evaluated using different salts in the binding solution at pH 8. SIGMA silica, ORGANON TEKNIKA silica, diatomaceous earth, Binding Matrix, and GLASSMILK were used for the following studies. The solid substrates were prepared as described in Example 1. The data presented in this example represents the consolidation of four experiments.

Each solid substrate and buffer combination was assayed once as follows. Rat liver total RNA (Biochain Institute, lot numbers A304057, A305062, or A306073; 15-26 μg of RNA at 2.5 mg/ml concentration in water) was added to a 1.5 ml microcentrifuge tube containing 0.45 ml of one of the following buffers: (1) 50 mM Tris HCl, pH 8, 4.75 M guanidine thiocyanate; (2) 50 mM Tris HCl, pH 8; 4.75 M guanidine hydrochloride; (3) 50 mM Tris HCl, pH 8; 4.75 M sodium chloride; or (4) 50 mM Tris HCl, pH 8; 4.75 M sodium iodide. Nucleic acid was incubated up to 5 minutes at ambient temperature in the buffered solution, with occasional mixing. Then each solid substrate (10 mg ORGANON TEKNIKA silica, 10 mg diatomaceous earth, 186 mg GLASSMILK, 58 mg BIO101 Binding Matrix) was added separately to each of the buffered nucleic acid solutions. Those mixtures were incubated 10 minutes at ambient temperature with occasional mixing.

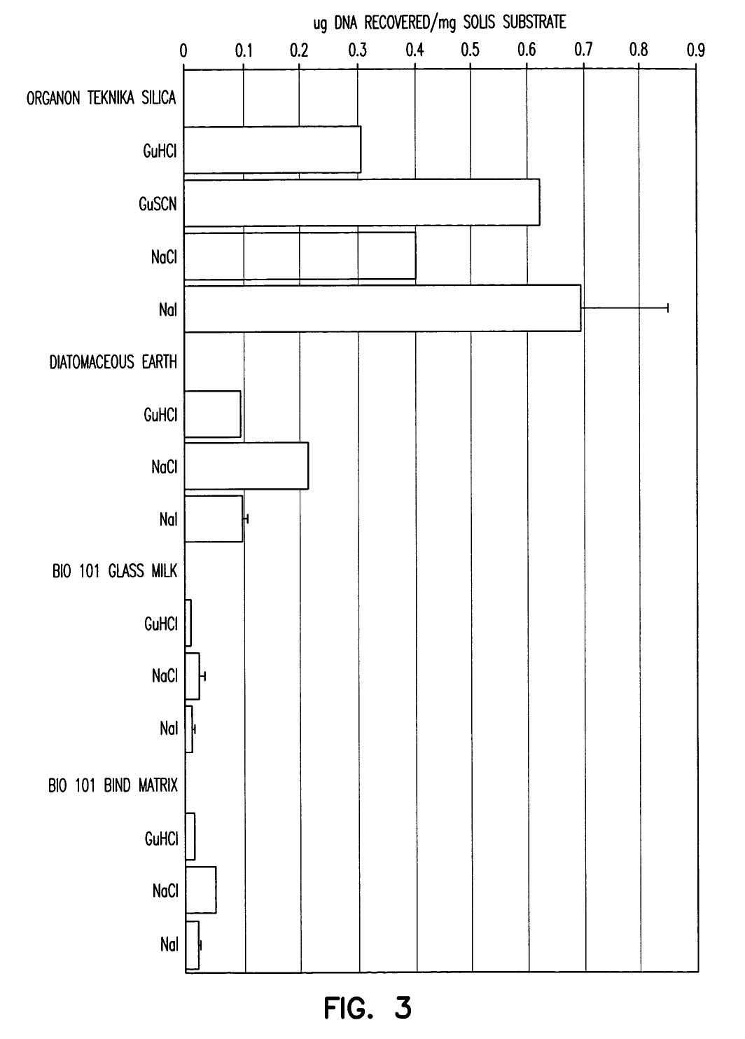

Washing of the solid substrates, elution of the nucleic acid, and quantitation of the eluted nucleic acid was as in Example 1. The results are shown in FIG. 3.

In contrast to the results found with DNA, recovery of RNA when bound to the solid substrates at pH 8 did not show a strong dependence on salt composition of the binding buffer. The selection of salt resulted in less than a threefold variation in the recovery of RNA for a particular substrate.

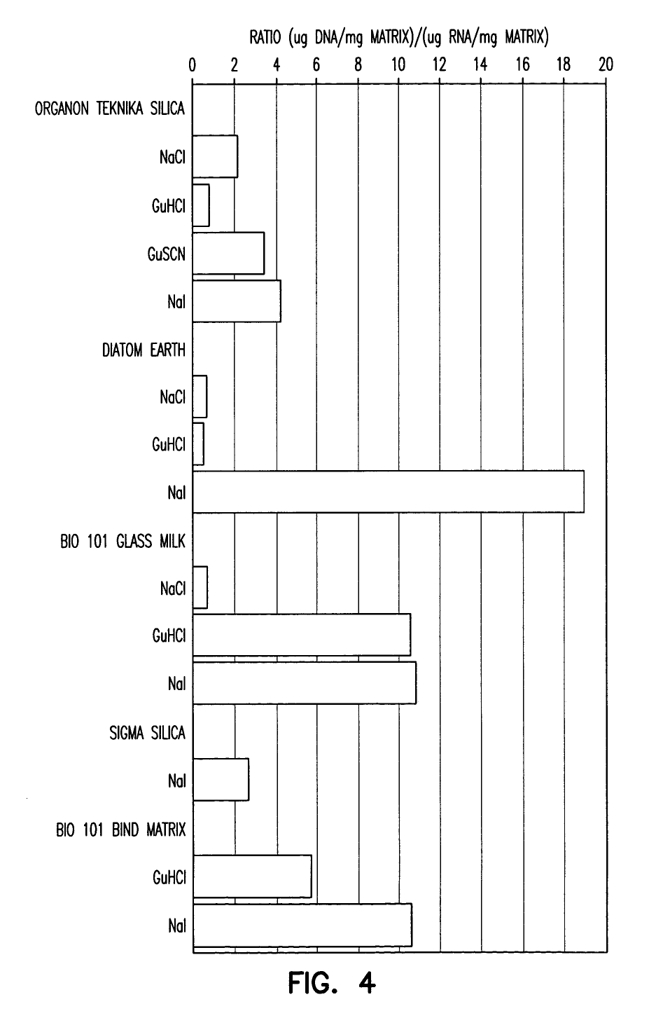

In order to compare the recovery of DNA to that of RNA under several of the conditions, the ratio of DNA recovered per mg solid substrate in the experiments of FIG. 1 to RNA recovered per mg solid substrate in the experiments of FIG. 3 is shown in FIG. 4. When binding at pH 8, many of the substrates showed a moderately higher recovery of DNA as compared to RNA. Selectivity for DNA recovery was greater with the larger anions thiocyanate and iodide in the binding solution (see FIG. 4).

### Example 4

DNA was bound to several solid substrates using buffers with different salt compositions and pH levels.

The following solid substrates used in this study were prepared as described in Example 1: ORGANON TEKNIKA silica, diatomaceous earth, and GLASSMILK. For these studies, sheared calf thymus DNA prepared as described in Example 1 was used as the source of genomic DNA.

Each solid substrate and buffer combination was assayed once as follows. Sheared calf thymus DNA (25 μg) was added to 1.5 ml microcentrifuge tubes containing 0.45 ml of one of the following buffers: (1) 50 mM MES, pH 6, 4.75 M guanidine thiocyanate; (2) 50 mM MES, pH 6.0, 4.75 M sodium chloride; (3) 50 mM MES, pH 6.0, 4.75 M sodium bromide; (4) 50 mM MES, pH 6.0, 4.75 M sodium iodide; (5) 50 mM Tris-HCl, pH 8, 4.75 M guanidine thiocyanate; (6) 50 mM Tris-HCl, pH 8, 4.75 M sodium chloride; (7) 50 mM Tris-HCl, pH 8, 4.75M sodium bromide; (8) 50 mM Tris-HCl, pH 8, 4.75 M sodium iodide; (9) 50 mM AMP, pH 10, 4.75 M guanidine thiocyanate; (10) 50 mM AMP, pH 10, 4.75 M sodium chloride; (11) 50 mM AMP, pH 10, 4.75 M sodium bromide; or (12) 50 mM AMP, pH 10, 4.75 M sodium iodide.

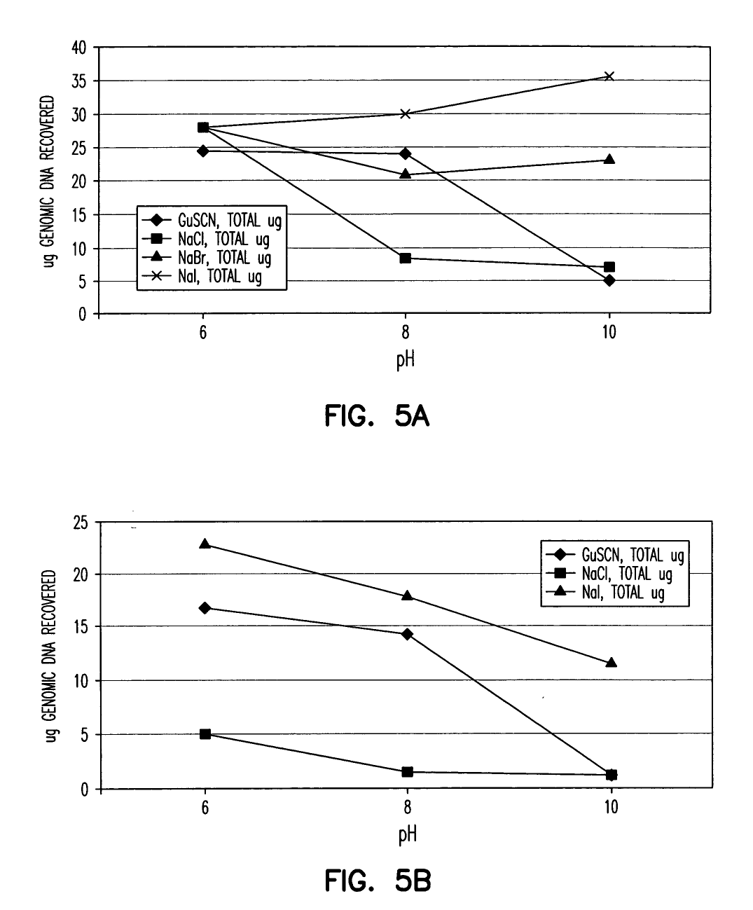

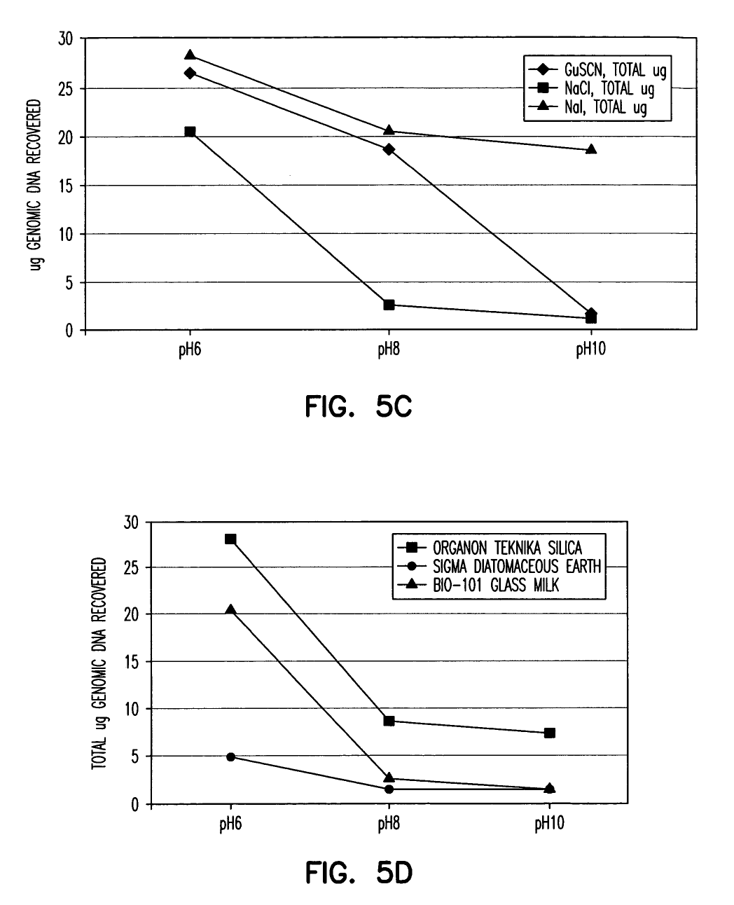

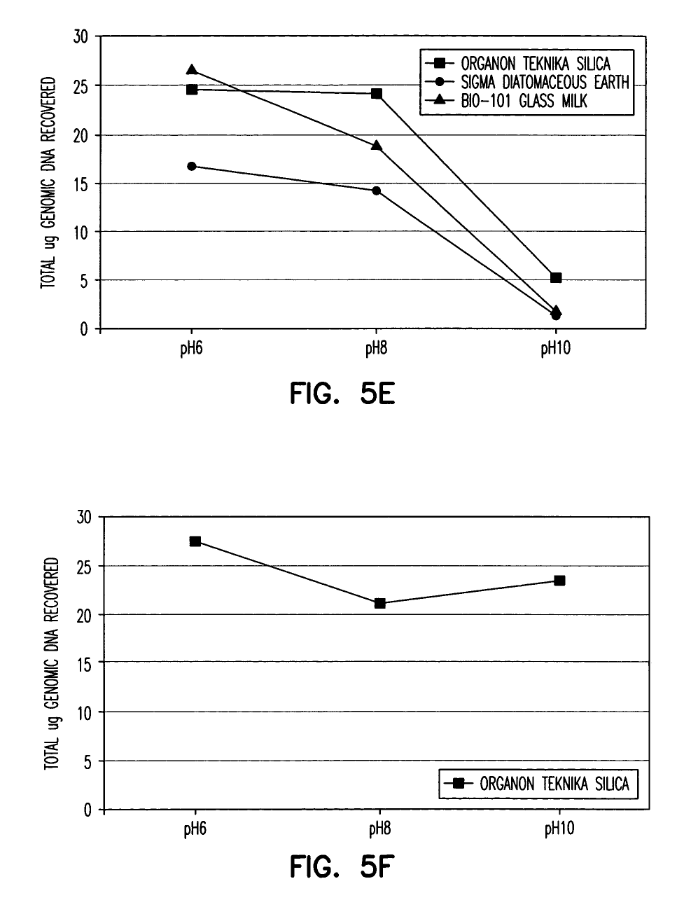

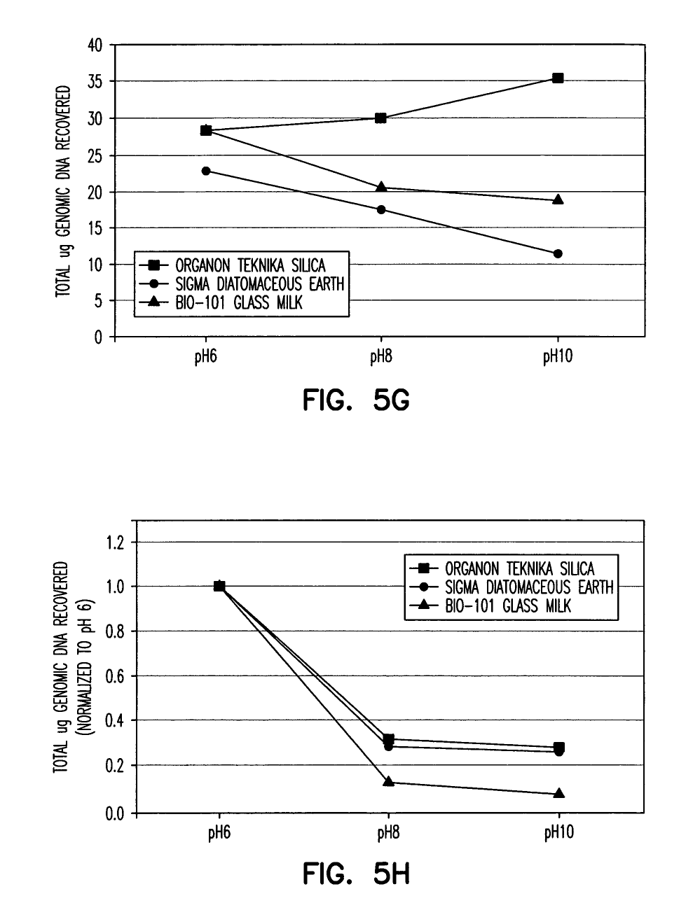

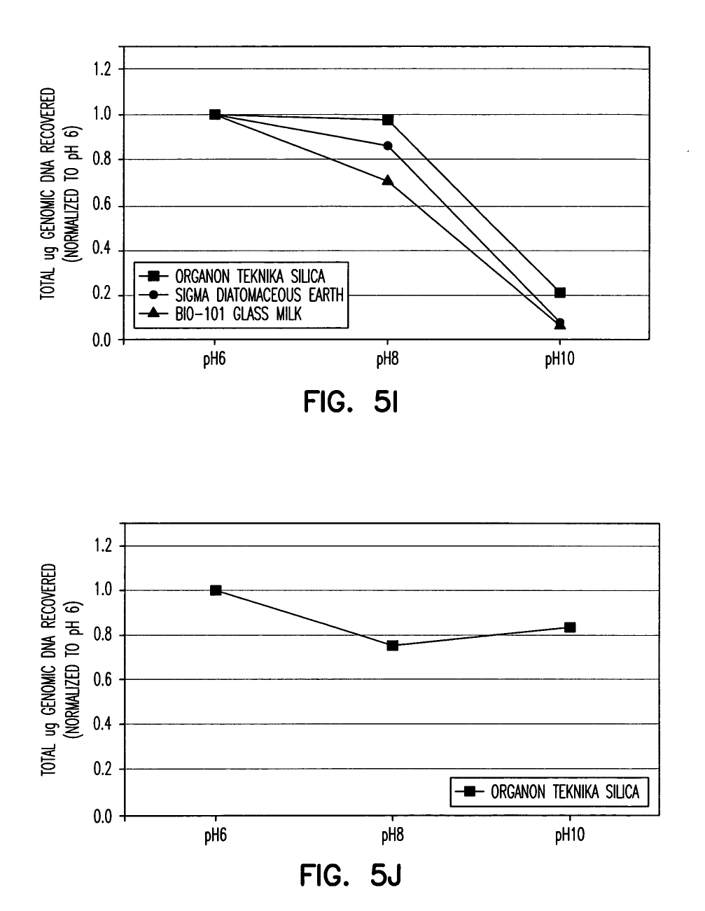

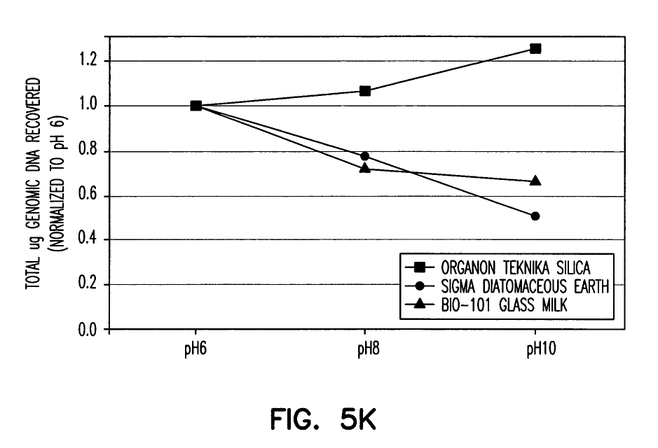

Nucleic acid was incubated for 5 to 10 minutes at ambient temperature in the buffered solution, sometimes with occasional mixing. The solid substrates (10 mg ORGANON TEKNIKA silica, 10 mg diatomaceous earth, 186 mg GLASSMILK) were added separately to each of the buffered nucleic acid solutions. The solutions were incubated for 10 minutes with the solid substrates at ambient temperature with occasional mixing. Washing of the particles, elution of the nucleic acid, and quantitation of the eluted nucleic acid was as in Example 1. Results for each set of experiments are shown in FIGS. 5(a)-(k).

Previous investigators showed that DNA binding to silica decreases as the pH of the buffer is increased above 7 (Melzak, Kathryn A. et al. (1996), Driving Forces for DNA Adsorption to Silica in Perchlorate Solutions, Journal of Colloid and Interface Science 181: 635-644). The results in this example showed that the salt composition influenced the effect of pH on DNA recovery. The results also demonstrated that different solid substrates recovered different amounts of nucleic acid, and that the choice of salt in the binding solution affected the amount of nucleic acid recovered. The source of the solid substrate altered the magnitude of the effect of the salt and the absolute amount of nucleic acid that was recovered. In general, the solid substrates recovered less DNA with binding at high pH than at lower pH. But the magnitude of this effect varied depending on the salt present in the binding solution. The amount of DNA recovered with silica-based solid substrates was approximately the same at various binding solution pH levels in the presence of bulky anions (FIGS. 5f and 5g). With chloride in the binding solution, however, recovery of DNA with binding to silica-based solid substrates was significantly reduced when the pH of the binding buffer was increased (FIG. 5d). Recovery of DNA from silica-based solid substrates decreased the least at higher binding solution pH levels in the presence of iodide.

### Example 5

The effect of pH and salt composition on RNA binding to solid substrates was also evaluated. The solid substrates Binding Matrix and GLASSMILK were prepared as described in Example 1. Rat liver total RNA was the source of RNA.

Each solid substrate and buffer combination was assayed once as follows. Rat liver total RNA (16 μg) was added to 1.5 ml microcentrifuge tubes containing 0.45 ml of one of the following buffers: (1) 50 mM Tris-HCl, pH 8, 4.75 M guanidine thiocyanate; (2) 50 mM Tris-HCl, pH 8, 4.75 M sodium chloride; (3) 50 mM Tris-HCl, pH 8, 4.75 M sodium iodide; (4) 50 mM MES, pH 6, 4.75 M guanidine thiocyanate; (5) 50 mM MES, pH 6, 4.75 M sodium iodide; (6) 50 mM MES, pH 6, 4.75 M sodium chloride; (7) 50 mM AMP, pH 10, 4.75 M guanidine thiocyanate; (8) 50 mM AMP, pH 10, 4.75 M sodium iodide; or (9) 50 mM AMP, pH 10, 4.75 M sodium chloride. Nucleic acid was incubated up to 5 minutes at ambient temperature in the buffered solution, with occasional mixing. Two solid substrates (10 mg ORGANON TEKNIKA and 186 mg GLASSMILK) were tested. The solid substrates were added separately to the buffered nucleic acid solutions (total of 18 reaction mixtures). The mixtures were incubated 10 minutes at ambient temperature with occasional mixing. Washing of the solid substrates, elution of the nucleic acid, and quantitation of the eluted nucleic acid was as in Example 1. The amount of RNA recovered was normalized to the amount of solid substrate initially added.

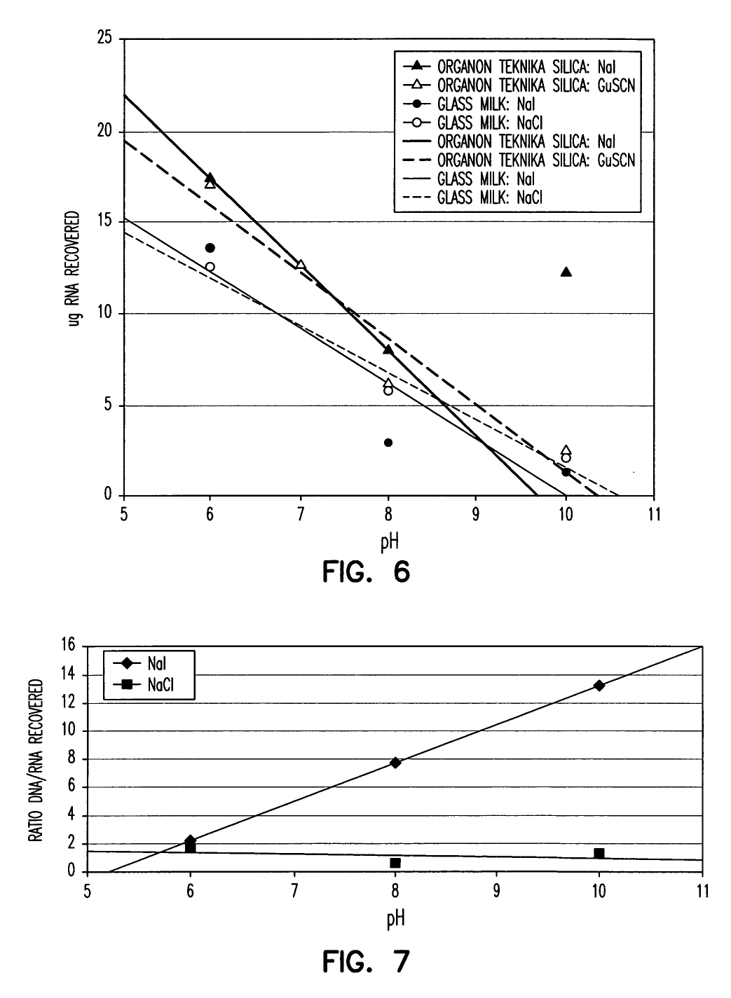

The results are shown in FIG. 6. Recovery of RNA from the two solid substrates evaluated varied considerably depending on the binding pH. RNA was recovered from the solid substrate in a significantly reduced amount when the pH of the binding solution was increased. This was true for all the different salt solutions and both solid substrates tested.

### Example 6

GLASSMILK was evaluated to determine conditions where it selectively bound DNA or RNA. The selectivity of GLASSMILK was evaluated further as a function of pH and ionic composition during binding. GLASSMILK was prepared as described in Example 1. Sheared calf thymus DNA was prepared as described in Example 1. Rat liver total RNA was the source of RNA.

Each nucleic acid and buffer combination was assayed once as follows. Either 30 μg of sheared calf thymus DNA or 16 μg of rat liver total RNA was added separately to 1.5 ml microcentrifuge tubes containing 0.45 ml of one of the following buffers: (1) 50 mM MES, pH 6.0, 4.75 M sodium chloride; (2) 50 mM MES, pH 6.0, 4.75M sodium iodide; (3) 50 mM Tris-HCl, pH 8, 4.75 M sodium chloride; (4) 50 mM Tris-HCl, pH 8, 4.75 M sodium iodide; (5) 50 mM AMP, pH 10, 4.75 M sodium iodide; or (6) 50 mM AMP, pH 10, 4.75 M sodium chloride. Thus, 12 containers were used. The solid substrate (GLASSMILK) (186 mg) was added to the buffered nucleic acid solutions and the mixtures were incubated 10 minutes at ambient temperature with occasional mixing. Washing of the solid substrate, elution of the nucleic acid, and quantitation of the eluted nucleic acid was as in Example 1.

The results are shown in FIG. 7. The GLASSMILK solid substrate preferentially bound DNA over RNA at higher pH values when NaI was the salt used in the binding solution. In contrast, the solid substrate did not preferentially bind DNA at increasing pH when the binding solution contained NaCl.

### Example 7

The effect of binding solution pH on DNA and RNA recovery with sodium iodide in the binding solution was evaluated with several solid substrates. SIGMA silica, DAVISIL Grade 643 silica gel, and Uniform Silica Microspheres (Bangs Laboratories, Inc. Catalog Code SSO5N, Inv # L0002188) were used for the following study. Prior to use, the silicon dioxide and DAVISIL Grade Silica Gel particles were prepared as follows. The solid substrates were washed once with water, once with 500 mM ammonium bifluoride, once with 100 mM nitric acid, twice with 100 mM ammonium hydroxide, twice with 300 mM ammonium hydroxide, once with ethanol, and nine times with water. All three solid substrates were prepared and stored as 200 mg/ml (20%) slurries in water.

Total rat liver RNA was the source of RNA. For genomic DNA, sheared calf thymus DNA was prepared as described in Example 1.

Each combination of solid substrate, nucleic acid, and buffer was assayed in triplicate. Either sheared calf thymus DNA (30 μg, addition of 50 μL of a 590 μg/mL stock) or total rat liver RNA (30 μg, addition of 50 μL of a 600 μg/mL stock) was added to 1.5 ml microcentrifuge tubes containing 50 μL of silica particles (10 mg) along with 0.45 ml of one of the following buffers: (1) 50 mM MES, pH 6.0, 4.8 M NaI; (2) 50 mM Hepes, pH 7.0, 4.8 M NaI; (3) 50 mM Tris, pH 8, 4.8 M NaI; (4) 50 mM Tris, pH 9, 4.8 M NaI; or (5) 50 mM AMP, pH 10, 4.8 M NaI. Therefore, a total of 90 tubes were used in the experiment. Nucleic acid was incubated with the solid substrate 5-30 minutes at ambient temperature in the buffered solution, with continuous mixing using a VORTEX GENIE-2 mixer at setting 7 (Scientific Industries).

Following binding, the solid substrates were centrifuged at 14,000 rpm for 1 minute and the supernatant removed. The solid substrates were subsequently washed four times with 1 mL of 70% ethanol. Following addition of 250 μL of 50 mM Tris, pH 9.0, the solid substrates were incubated for 5-6 minutes at 56° C. with continuous shaking (1400 rpm) on an EPPENDORF THERMOMIXER R mixer. The solid substrates were centrifuged at 14,000 rpm for 1 minute and the supernatant containing eluted nucleic acid was collected. In order to detect the presence of residual nucleic acid still bound to the solid substrate, 250 μL of 100 mM NaOH was added. The solid substrates were incubated for 5-70 minutes at 56° C. with continuous shaking (1400 rpm) on an EPPENDORF THERMOMIXER R mixer, and the supernatant was collected, which contained the eluted residual nucleic acid. The amount of nucleic acid in each fraction was quantified spectrophotometrically.

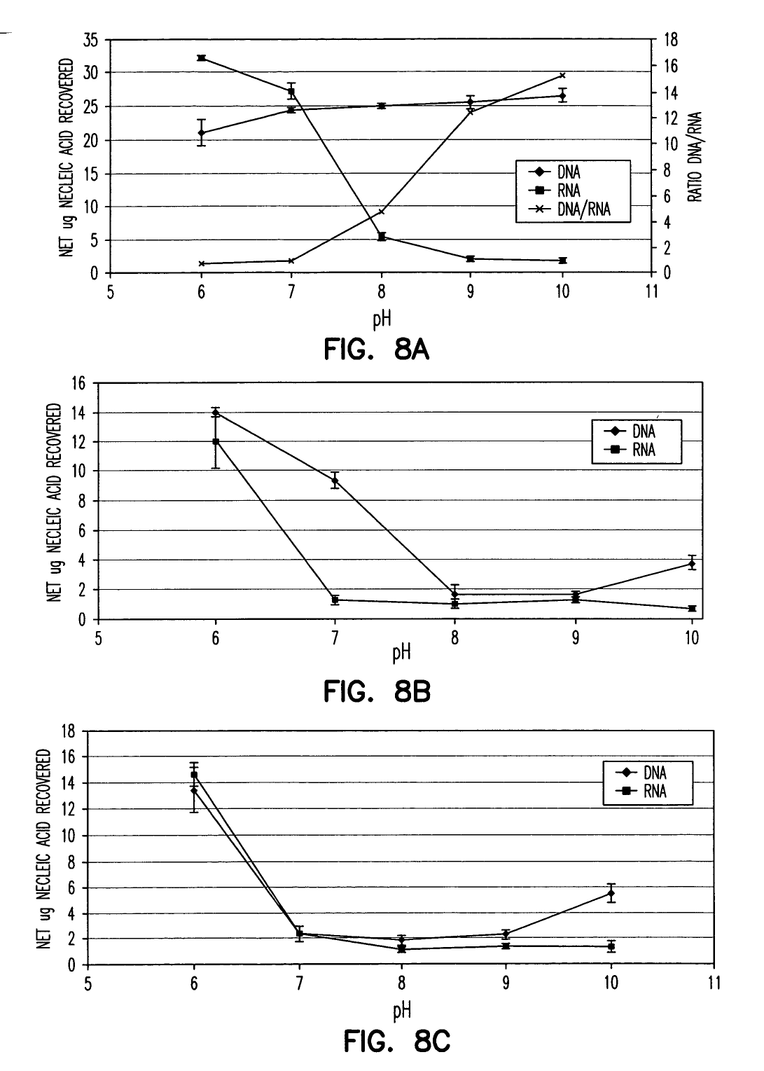

The results are shown in FIGS. 8(a)-(c). SIGMA silica had an increased selectivity for DNA binding over RNA binding at alkaline pH in these experiments with sodium iodide. DNA was recovered from SIGMA silica in approximately the same quantity regardless of the binding pH, while RNA was recovered in dramatically lower amounts when higher binding pHs were used. Thus, DNA selectivity was seen at an alkaline pH in the presence of NaI. The other solid substrates tested—BANGS Uniform Silica Microspheres and DAVISIL silica gel—also showed selectivity for recovery of DNA over RNA, although the binding buffer pH range for the selectivity was narrower than that found for SIGMA silica.

### Example 8

The effect of pH on the selective recovery of DNA vs. RNA with an additional set of solid substrates was examined. Sodium iodide was the binding salt in this experiment. ORGANON TEKNIKA silica, diatomaceous earth, SIGMA silica, Binding Matrix, and GLASSMILK were prepared as described in Example 1. Binding was carried out in the presence of sheared calf thymus DNA (as described in Example 1) or rat liver total RNA. Binding was performed in the following binding buffers: (1) 50 mM Tris-HCl, pH 8, 4.75 M NaI; or (2) 50 mM AMP, pH 10, 4.75 M NaI.

Either sheared calf thymus DNA (30 μg) or total rat liver RNA (16 or 26 μg) was added to 1.5 ml microcentrifuge tubes containing 4.5 ml of one of the following buffers: (1) 50 mM Tris-HCl, pH 8, 4.75 M NaI; or (2) 50 mM AMP, pH 10, 4.75M NaI.

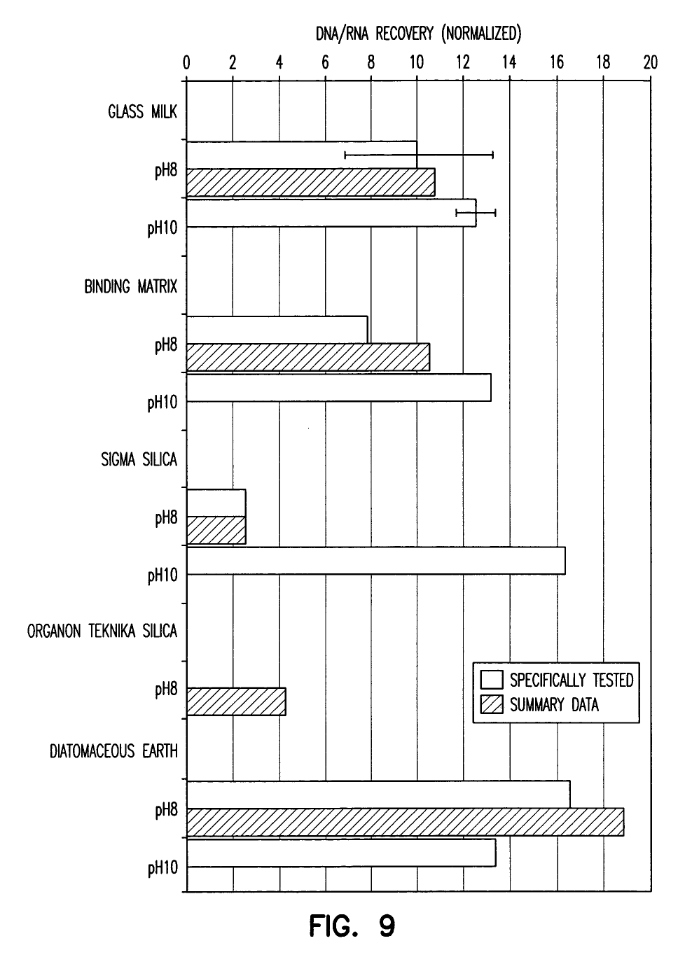

A single solid substrate (186 mg GLASSMILK, 57.6 mg BIO101 Binding Matrix, 10 mg SIGMA silica, 10 mg diatomaceous earth) was added to the RNA or DNA in the different buffers. FIG. 9 gives the results for each of the experiments (“Specifically Tested”). In addition, FIG. 9 shows the values displayed in FIG. 4 for comparison (“Summary Data”). The data obtained for GLASSMILK and Binding Matrix at pH 8 are compilations of two experiments for each. Thus, there were 10 containers with each of the possible combinations of solid substrate and buffer for the RNA, and 10 containers with each of the possible combinations of solid substrate and buffer for the DNA. The mixtures were incubated for 10 minutes at ambient temperature with occasional mixing. Washing of the solid substrate, elution of the nucleic acid, and quantitation of the eluted nucleic acid was as in Example 1.

The results in FIG. 9 indicate that each of the solid substrates examined had a specificity for DNA recovery over RNA recovery using iodide as the bulky anion at the pHs tested. GLASSMILK, Binding Matrix and SIGMA silica all had a greater selectivity for DNA over RNA at pH 10 than at pH 8. Diatomaceous earth had a greater selectivity for DNA over RNA at pH 8.

### Example 9

To measure the difference between the binding of DNA and RNA to silica, and the level of saturation, the following experiment was performed. The SIGMA silica solid substrate was prepared as described in Example 1. The nucleic acids studied were sheared calf thymus DNA (prepared as described in Example 1) and total rat liver RNA.

Sheared calf thymus DNA (126 μg, 60 μg, 30 μg, 15 μg, or 5 μg) or total rat liver RNA (125 μg, 60 μg, 30 μg, 15 μg, or 5 μg) was added in 50 μL to microcentrifuge tubes (1.5 ml) containing 450 μl of binding buffer (50 mM Tris, pH 8, 4.8 M sodium iodide), and 10 mg of SIGMA silica (prepared as described in Example 1). The work was performed in triplicate, so there were 15 containers for the five different amounts of DNA and 15 containers for the five different amounts of RNA. The samples were incubated at ambient temperature for 5-10 minutes on a VORTEX GENIE-2 mixer at setting 7 (Scientific Industries). Washing of the solid substrate, elution of the nucleic acid, and quantitation of the eluted nucleic acid was as in Example 1.

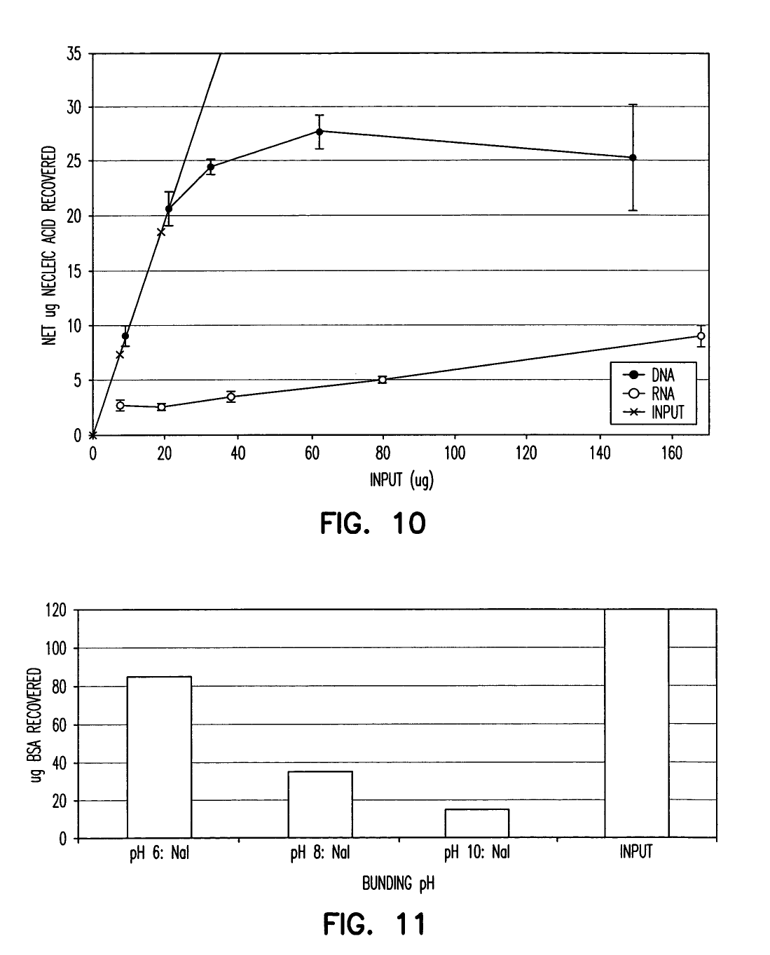

The results are shown in FIG. 10. Under the binding conditions used in this study, 10 mg of the silica particles had a capacity of ˜25 μg for genomic DNA. Binding of DNA at concentrations below saturation of the solid substrate was efficient. Virtually all of the added DNA was bound and recovered from the silica-based solid substrate. In contrast, binding of RNA under these conditions was inefficient even when the amount added was high. In fact, the low amount of nucleic acid recovered from the RNA samples may represent contaminating DNA since the spectrophotometric measurements cannot distinguish DNA from RNA.

This demonstrates that under selective binding conditions, at pH 8 in the presence of 4.8 M sodium iodide, the capacity of the SIGMA silica solid substrate for DNA was about 25 μg DNA/10 mg solid substrate.

### Example 10

In order to test whether the silica solid substrate is actually selective for DNA over RNA, or whether the results from Example 9 were instead an artifact resulting from RNA degradation due to the alkalinity of the binding buffer, the following experiments were performed. RNA was incubated either at pH 6 or pH 10 prior to binding to SIGMA silica under conditions compatible with RNA binding as follows. Total rat liver RNA (25 μg in a total volume of 10 μL) was incubated, in duplicate, in 100 μL of either 50 mM AMP, pH 10, containing 4.8 M NaI; or 50 mM MES, pH 6, containing 4.8 M NaI at ambient temperature for 5, 10, 15, 30, or 60 minutes. At the end of the indicated time, 1 mL of 50 mM MES, pH 6, containing 4.8 M NaI was added to each tube in order to render conditions compatible with RNA binding to silica. The reactions were mixed and 10 mg of SIGMA silica (prepared as described in Example 1) in a total volume of 50 μL was added. The samples were incubated at ambient temperature for 10 minutes on a VORTEX GENIE-2 mixer at setting 7 (Scientific Industries). Washing of the solid substrate, elution of the nucleic acid, and quantitation of the eluted nucleic acid was as in Example 1.

The half-life of RNA was found to be 86 minutes at pH 10 and 260 minutes at pH 6. Based on these half-life values, only 7.7% of the added RNA would be expected to degrade during a 10 minute binding incubation at pH 10, and only 2.6% of added RNA would be expected to degrade during a 10 minute incubation at pH 6. Therefore, this experiment confirms that the results in Example 9 were due to selective binding of DNA as compared to RNA by the silica solid substrate, and not merely due to the degradation of RNA.

### Example 11

The effect of pH on protein binding to SIGMA silica was examined. Each condition was assayed once. Purified bovine serum albumin (1 mg, 100 μl of a 10 mg/ml solution in water, New England Biolabs, Lot 938) was added to 1.5 ml microcentrifuge tubes containing 1 ml of: (1) 50 mM MES, pH 6.0, 4.75 M NaI; (2) 50 mM Tris-HCl, pH 8, 4.75 M NaI; or (3) 50 mM AMP, pH 10, 4.75 M NaI. The silica solid substrate (10 mg) was added to the buffered protein solutions and the mixtures were incubated at ambient temperature with occasional mixing. Washing of the solid substrate, elution of the nucleic acid, and quantitation of the eluted nucleic acid was as in Example 7. Protein was eluted in the same manner, and quantified by absorbance at 280 nm using the conversion factor of 1 O.D._{280 nm}=1 mg/ml protein.

The results are shown in FIG. 11. Less protein was recovered from the silica as the pH of the binding buffer was increased.

### Example 12

The effect of binding solution salt composition on DNA and RNA recovery from silica was examined at alkaline binding pH. SIGMA silica was prepared as described in Example 1. Sheared calf thymus DNA was prepared according to Example 1. Rat liver total RNA was the source of RNA.

Each nucleic acid and buffer combination was assayed once. Either 25 μg (50 μl of a 0.5 mg/ml solution in water) sheared calf thymus DNA or 25 μg (10 μl of a 2.5 mg/ml solution in water) rat liver total RNA was added to 1.5 ml microcentrifuge tubes containing 0.5 ml of one of the following buffers: (1) 50 mM AMP, pH 10, 3.65 M lithium chloride; (2) 50 mM AMP, pH 10, 3.65 M lithium bromide; (3) 50 mM AMP, pH 10, 3.65 M lithium iodide; (4) 50 mM AMP, pH 10, 3.65 M sodium chloride; (5) 50 mM AMP, pH 10, 3.65 M sodium bromide; (6) 50 mM AMP, pH 10, 3.65 M sodium iodide; (7) 50 mM AMP, pH 10, 3.65 M potassium chloride; (8) 50 mM AMP, pH 10, 3.65 M potassium bromide; or (9) 50 mM AMP, pH 10, 3.65 M potassium iodide. Because of the lower solubility of some of the salts and the desire to maintain equivalent concentrations of all salts examined, each salt was adjusted to 3.65 M, in contrast to previous experiments.

SIGMA silica (10 mg) was added to each of the 18 buffered nucleic acid solutions and the mixtures were incubated for 10 minutes at ambient temperature with occasional mixing. Washing of the solid substrate, elution of the nucleic acid, and quantitation of the eluted nucleic acid was as in Example 7.

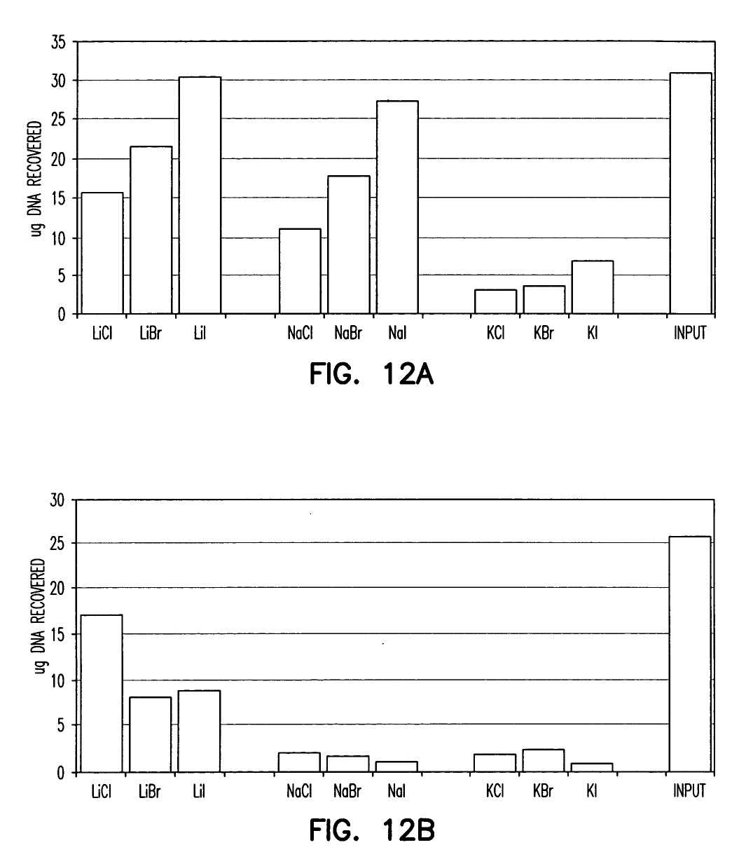

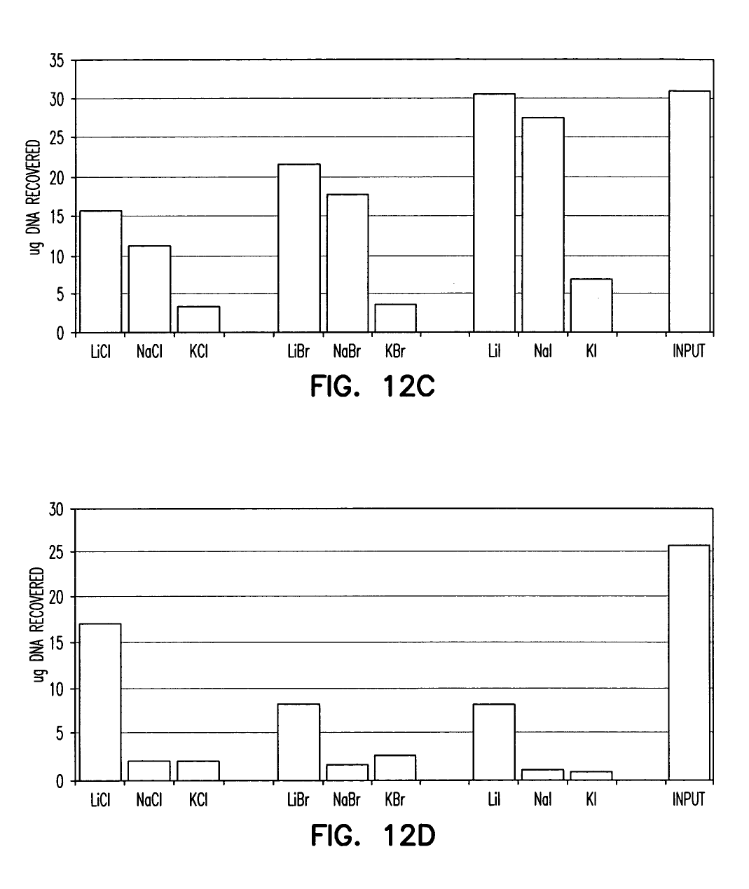

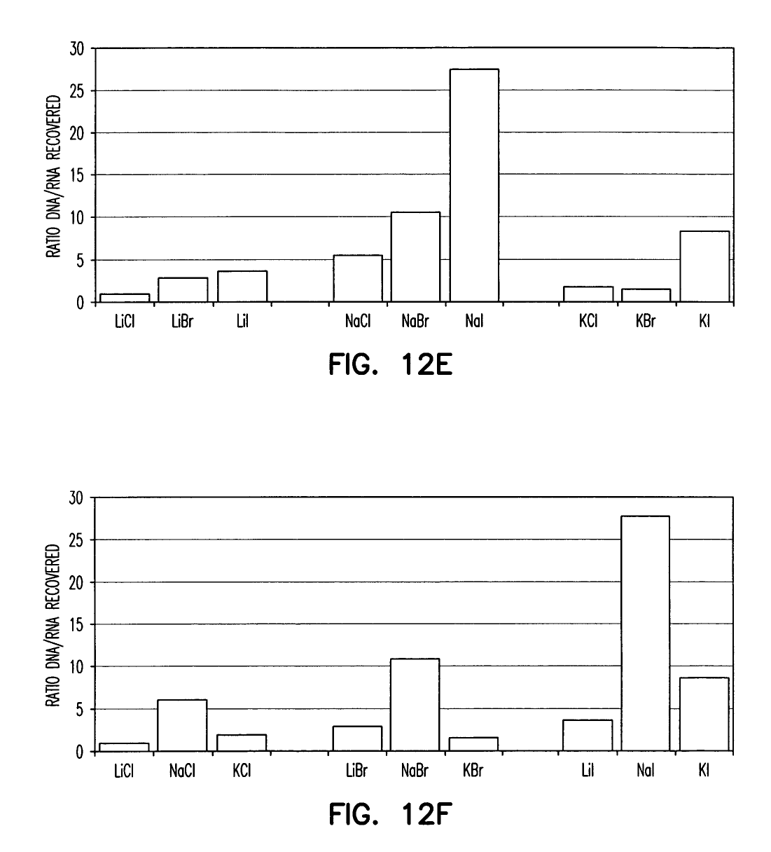

The effect of cations and anions on nucleic acid recovery from the silica solid substrate is shown in FIGS. 12(a)-(f). FIGS. 12(a) and (c) provide the data obtained from experiments using DNA. FIGS. 12(b) and (d) provide the results of the RNA experiments. FIG. 12(e) shows the ratio of DNA/RNA recovery grouped by cation, while FIG. 12(f) shows the ratio of DNA/RNA recovery grouped by anion.

At a binding pH of 10, recovery of DNA from the silica was influenced by the anion present in the binding solution. DNA recovered from silica increased as the monovalent anion radius was increased. The radius of the monovalent cation influenced the magnitude of that effect. In contrast, the recovery of RNA from silica showed a tendency to decrease as the anion radius was increased. Decreasing the size of the cation increased the binding capacity of the silica solid substrate for both nucleic acid species.

Selectivity for DNA over RNA recovery from a solid substrate is improved with certain selections of salt composition in the binding buffer. These results (FIG. 12(e)) showed that the larger the anion, the greater the selectivity for DNA over RNA. There was no correlation between the cation radius and selectivity for DNA over RNA recovery (FIG. 12(f)).

Lithium and sodium cations both allowed high capacity for DNA, while potassium decreased the binding capacity for DNA of the silica. In sodium salts, the solid substrate had a higher selectivity for DNA over RNA than in lithium salts.

### Example 13

In this Example, the ability of high concentrations of RNA to inhibit binding of genomic DNA was examined. SIGMA silica and genomic DNA were prepared as described in Example 1. Rat liver total RNA was the source of RNA.

Each condition was assayed once. Five 10 μl mixtures of RNA and DNA at RNA:DNA ratios of 1:1 (15 μg:15 μg), 10:1 (15 μg:1 μg), 30:1 (15 μg:0.5 μg), or 100:1 (20 μg:0.2 μg), were added to 1.5 ml microcentrifuge tubes containing 0.45 ml of one of the following buffers: (1) 50 mM AMP, pH 10, 4.75 M NaI; or (2) 50 mM MES, pH 6.0, 4.75 M NaI. The SIGMA silica solid substrate (10 mg) was added to each of the 10 buffered nucleic acid solution combinations and the mixtures were incubated for 10 minutes at ambient temperature with occasional mixing. Washing of the solid substrate, elution of the nucleic acid, and quantitation of the eluted nucleic acid was as in Example 7.

Recovery of nucleic acid was visualized by agarose gel electrophoresis. Electrophoresis was performed on 2 μl of the nucleic acid stock mixes or 5 μl of the isolated eluate. Samples were electrophoresed through a 1% SEAKEM (Teknova), 0.5 μg/ml ethidium bromide gel using 1×TBE, 0.5 μg/ml ethidium bromide buffer (Bio-Rad), at 8 V/cm for 30 minutes to 1 hour. Ethidium-stained material was visualized and photographed under short wave ultra-violet light. Molecular weight markers consisted of an AMPLISIZE DNA molecular weight standard (Bio-Rad) and an RNA ladder (Gibco BRL).

The results showed that at a binding pH of 10 in NaI, silica was at least 40-fold more selective for recovery of DNA over RNA. At pH 6, there was nearly complete capture of the added RNA, but even at the highest level of added RNA (20 μg), no detectable RNA was recovered at pH 10. At pH 6, little DNA was recovered from the sample containing 15 μg RNA and 1.5 μg DNA. Thus, in the absence of selectivity, the binding of RNA to the solid-phase reduced the recovery of DNA from the sample. In contrast, when 0.5 μg of genomic DNA was added, detectable DNA was recovered with binding at pH 10, despite the presence of 15 μg of RNA in the sample. Therefore, at pH 10, in the presence of sodium iodide, silica showed greater that 40-fold greater selectivity for DNA over RNA. The lack of binding of RNA at the higher pH allows for controlled recovery of DNA for subsequent analyses.

### Example 14

To further evaluate whether high levels of RNA would compete with DNA for binding to a solid substrate, high levels of RNA were mixed with genomic DNA and bound under both nonselective and selective conditions. SIGMA silica and sheared genomic DNA were prepared as described in Example 1. Rat liver total RNA was the source of RNA.

Each nucleic acid and buffer combination was assayed twice. Either (a) 5 μg sheared calf thymus DNA (10 μl of 0.5 mg/ml) and 5 μg (2 μl of 2.5 mg/ml) of rat liver total RNA, or (b) 5 μg sheared calf thymus DNA (10 μl of 0.5 mg/ml) and 50 μg (20 μl of 2.5 mg/ml) of rat liver total RNA were added and mixed in 1.5 ml microcentrifuge tubes containing 0.45 ml of one of the following buffers: (1) 50 mM AMP, pH 10, 3.5 M NaI; or (2) 50 mM MES, pH 6.0, 3.5 M NaI. The solutions were incubated at ambient temperature for 5 minutes.

The SIGMA silica solid substrate (10 mg) was added to each of the four buffered nucleic acid solution combinations and the mixtures were incubated for 10 minutes at ambient temperature with occasional mixing. Following the binding incubation, the solid substrate was centrifuged (15,800×g, 1 minute), and washed three times with 0.5 ml of 70% ethanol. The bound nucleic acid was eluted with 100 μl of 10 mM Tris, pH 9, for 5 minutes at 56° C. with constant mixing and the eluted nucleic acid was collected.

The recovery of nucleic acid was visualized by agarose gel electrophoresis. Of the isolation eluate, 10 μl was electrophoresed through a 0.8% agarose gel as described in Example 13. Ethidium-stained material was visualized and photographed under a short wave ultraviolet light.

Even at excess RNA concentrations, minimal RNA was isolated under the DNA selective conditions (pH 10, 3.5 M NaI). The data showed that the amount of RNA contamination was lower than the limit of detection when using DNA selective conditions. The same amount of DNA appeared to be recovered whether 5 or 50 μg of RNA was present in the mixture. Under nonselective conditions (pH 6, 3.5 M NaI), when a high concentration of nucleic acid is present, the limited number of nucleic acid binding sites on the solid substrate reduced overall DNA recovery.

### Example 15

The ability of a silica solid substrate to recover genomic DNA from a complex biological mixture (whole blood) was evaluated.

SIGMA silica was prepared as described in Example 1. Each condition was assayed once. Either 25 μl or 100 μl of whole human blood (Blood Centers of the Pacific) was added to 1.5 ml microcentrifuge tubes containing either 0.25 ml or 0.9 ml (respectively) of one of the following buffers: (1) 50 mM MES, pH 6.0, 4.75 M NaI, or (2) 50 mM AMP, pH 10, 4.75 M NaI. The mixtures were incubated 10 minutes at ambient temperature in the buffered solution, with occasional mixing. These conditions lysed cells, releasing nucleic acid.

The SIGMA silica solid substrate (10 mg) was added to the four buffered nucleic acid solution combinations, and the mixtures were incubated 10 minutes at ambient temperature with occasional mixing. Following the binding incubation, the silica solid substrate was centrifuged (15,800×g, 1 minute), and washed twice with 0.5 ml of the binding buffer that had been used for the binding incubation. Subsequently, the silica solid substrate was washed four times with 70% ethanol. The bound nucleic acid was eluted with 50 μl of 10 mM Tris, pH 9, for 5 minutes at 56° C. with constant mixing and the eluted nucleic acid was collected.

The recovery of nucleic acid was visualized by agarose gel electrophoresis. Either 2 μl or 10 μl of the eluate was electrophoresed through a 1.0% agarose gel as described in Example 13. Ethidium-stained material was visualized and photographed under short-wave ultra-violet light.

When silica was added to whole blood in NaI-containing buffer at pH 6, the silica particles clumped, suggesting that protein also adsorbed to the particles. As a result, DNA recovery was poor. In contrast, the silica remained in suspension when added to blood at pH 10 because the protein from whole blood did not bind to the silica solid substrate at the higher pH. The amount of DNA bound at pH 10 was high. Thus, this experiment showed that in the absence of protein adsorption to the silica solid substrate, DNA recovery was high.

### Example 16

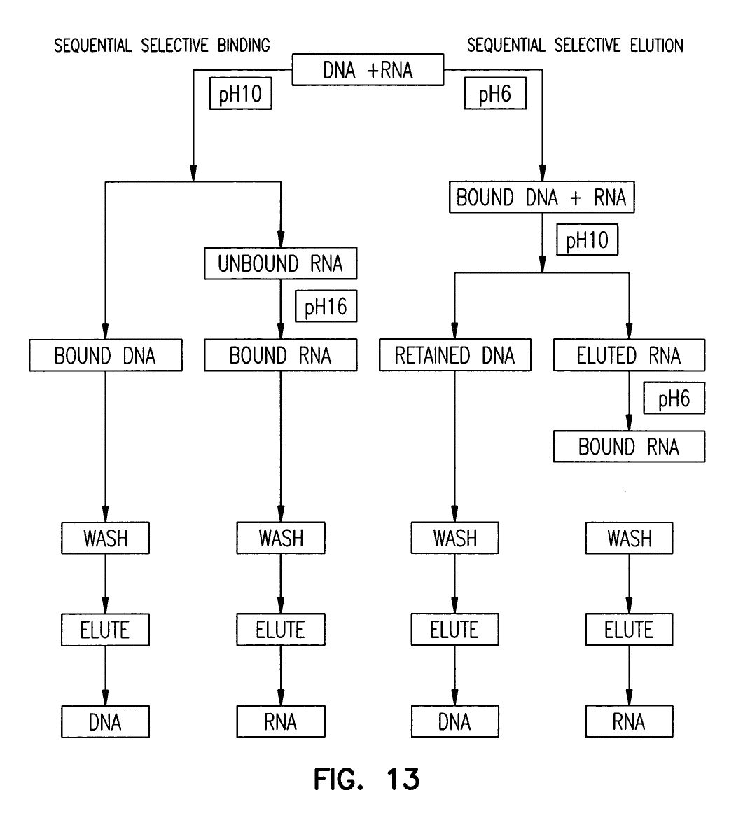

In certain embodiments, both DNA and RNA can be isolated from a sample mixture in pure form by sequential selective binding to a solid substrate. An exemplary sequential selective binding flow chart is shown in FIG. 13. In the sequential selective binding protocol, the sample is contacted with the solid substrate under conditions compatible with selective DNA binding. The solid substrate containing the bound DNA is separated from the unbound material. The conditions in the unbound fraction are then adjusted to those compatible with RNA binding to the solid substrate. A second solid substrate aliquot is added to this second fraction to adsorb the RNA. DNA and RNA are then isolated from the respective solid substrate aliquots.

To evaluate an embodiment of sequential selective binding of DNA and RNA to solid substrates, a mixture of the two was processed under sequential selective binding conditions. First, the DNA was bound to the solid substrate under DNA selective conditions and then the unbound RNA fraction was removed and bound to a second aliquot of the same solid substrate using nonselective binding conditions. SIGMA silica and sheared genomic DNA were prepared as described in Example 1. The RNA was rat liver total RNA (Biochain Institute).

Each nucleic acid and buffer combination was assayed twice. Five μg DNA, 5 μg RNA, and a mixture of 5 μg DNA and 5 μg RNA were added to separate 1.5 ml microcentrifuge tubes containing 50 mM AMP, pH 10, 3.5 M NaI; 50 mM MES, pH 6, 3.5 M NaI; and 50 mM AMP, pH 10, 3.5 M NaI respectively. The buffered solutions were incubated for 5 minutes at ambient temperature. Ten mg of the silica solid substrate was added to each of the 6 buffered nucleic acid solution combinations and the mixtures were incubated for 10 minutes at ambient temperature with occasional mixing.

Following the binding incubation, the silica solid substrate was centrifuged (15,800×g, 1 minute) and washed 3 times with 0.5 ml of 70% ethanol. Once the ethanol was removed, the solid substrate was allowed to dry at ambient temperature at least 10 minutes.

The supernatants from the binding reactions of the mixture of DNA and RNA were taken and added to separate 1.5 ml microcentrifuge tubes containing 1 mL of 50 mM MES, pH 6, 3.5 M NaI. To these tubes was added 10 mg of the solid substrate, and the mixtures were incubated for 10 minutes at ambient temperature with occasional mixing.

Following the binding incubation, the silica solid substrate was centrifuged (15,800×g, 1 minute) and washed 3 times with 0.5 mL of 70% ethanol. Once the ethanol was removed, the solid substrate was allowed to dry at ambient temperature at least 10 minutes.

The bound nucleic acid was eluted from the solid substrate with 0.1 mL of 10 mM Tris-HCl, pH 9, for 5 minutes at 56° C. with constant mixing and the eluted nucleic acid was collected.

The recovery of nucleic acid was visualized by agarose gel electrophoresis. Electrophoresis was performed on 10 μL of each isolated eluate. Samples were electrophoresed through a 0.8% agarose, 0.5 μg/mL ethidium bromide gel using 1×TBE, 0.5 μg/mL ethidium bromide buffer (Bio-Rad), at 7V/cm for 30 minutes to 1 hour. Ethidium-stained material was visualized and photographed under short wave ultraviolet light. Molecular weight markers consisted of High Molecular Weight DNA Markers (Gibco BRL).

The gel showed that both RNA and DNA were pure following the sequential selective binding isolation from a mixture of both. No RNA could be visualized in the isolated DNA fraction, and no DNA could be visualized in the isolated RNA fraction. The gel also showed that the amount of DNA recovered, and thus the binding capacity of the solid substrate for DNA, under selective binding conditions was not affected by the presence of RNA.

### Example 17

In certain embodiments, both DNA and RNA can be isolated from a sample mixture by first binding both species to a solid substrate and sequentially releasing each nucleic acid type under the appropriate conditions (sequential selective elution). An exemplary sequential selective elution is shown in FIG. 13. In sequential selective elution, the sample is contacted with the solid substrate under conditions where the substrate binds both DNA and RNA. After washing the solid substrate of contaminants, the RNA is released under conditions where the solid substrate binds only DNA. The solid substrate is removed and the DNA and RNA are subsequently purified from both fractions.

To evaluate an embodiment of sequential selective elution, the following experiments were performed. The RNA is eluted under conditions which bind only DNA, and the DNA is subsequently eluted using a low ionic strength buffer. SIGMA silica and sheared genomic DNA were prepared as described in Example 1. Rat liver total RNA (Biochain Institute) was the source of RNA.

Each nucleic acid and buffer combination was assayed twice, and the DNA and RNA were processed as follows. Either (1) 10 μg sheared calf thymus DNA, or (2) 10 μg rat liver total RNA, or (3) 10 μg sheared calf thymus DNA and 10 μg rat liver total RNA were added to 1.5 ml microcentrifuge tubes containing 0.2 ml 50 mM MES, pH 6, 3.5 M sodium iodide, and incubated five minutes at ambient temperature with occasional mixing. The SIGMA silica solid substrate (10 mg) was added to each of the buffered nucleic acid solutions and the mixtures were incubated for 10 minutes at ambient temperature with mixing.

Following the binding incubation, the solid substrate, with the exception of one replicate solid substrate that bound both DNA and RNA, was centrifuged (15,800×g, 1 minute) and the bound nucleic acid was eluted with 0.2 mL 10 mM Tris, pH 9 for 5 minutes at 56° C. with constant mixing, and the eluted nucleic acid was collected.

One aliquot of solid substrate that bound both DNA and RNA was washed with 0.2 ml 50 mM AMP, pH 10, 3.5 M NaI, for 5 minutes at ambient temperature with occasional mixing, and the RNA was eluted and collected. The solid substrate was then washed with 0.2 ml 10 mM Tris, pH 9, for 5 minutes at 56° C. with constant mixing, and the bound DNA was eluted and collected.

The recovery of nucleic acid was visualized by agarose gel electrophoresis. Ten μl of each eluate was electrophoresed through a 1%, 0.5 μg/mL ethidium bromide gel using 1×TBE, 0.5 μg/mL ethidium bromide buffer (BIO-RAD), at 7 V/cm for 30 minutes to 1 hour. Ethidium-stained material was visualized and photographed under short wave ultra-violet light. Molecular weight markers consisted of High Molecular Weight DNA Markers (GIBCO BRL). The results showed that the low-salt Tris wash released substantially all of both RNA and DNA from the silica solid substrate. After both DNA and RNA were bound to the silica solid substrate, washing the substrate with the pH 10, NaI solution gave almost complete release of RNA with no detectable release of DNA. Subsequently washing the solid substrate with the low-salt Tris solution released DNA with little or no RNA that was visible on the gel. Thus, sequential selective elution successfully isolated pure RNA and pure DNA in good yield from a mixture of both nucleic acids.

### Example 18

To determine the relationship between surface area of a solid substrate and binding capacity, binding of DNA to Uniform Silica Microspheres (Bangs Laboratories, Inc.) in a range of sizes was tested. The following sizes of Uniform Silica Microspheres were examined: 0.14 μm diameter (Bangs, Laboratories, Inc. Catalog Code SS02N, Lot 4640); 0.4 μm diameter (Catalog Code SS02N, Lot 4195); 0.64 μm diameter (Catalog Code SS03N, Lot 4322); 0.81 μm diameter (Catalog Code SS03N, Lot 4183); 0.9 μm diameter (Catalog Code SS039N, Lot 4299); 1.28 μm diameter (Catalog Code SS04N, Lot 4253); 1.5 μm diameter (Catalog Code SS04N, Lot 4258); 1.6 μm diameter (Catalog Code SS04N, Lot 4599) 2.28 μm diameter (Catalog Code SS05N, Lot 4321, Inventory #L0002188); 2.3 μm diameter (Catalog Code SS05N, Lot 4186); 3 μm diameter (Catalog Code SS05N, Lot 4181); and 5.17 μm diameter (Catalog Code SS05N, Lot 4364, Inventory L000321A). The following amount of each bead size was added during the binding reaction: 0.14 μm diameter (50 μL=5.4 mg); 0.4 μm diameter (50 μL=5.0 mg); 0.64 μm diameter (50 μL=5.2 mg); 0.81 μm diameter (50 μL=5.25 mg); 0.9 μm diameter (50 μL=4.75; 1.28 mg); 1.28 μm diameter (100 μL=10.02 mg); 1.5 μm diameter (100 μL=10.2 mg); 1.6 μm diameter (100 μL=9.6 mg) 2.28 μm diameter (100 μL=10.1 mg); 2.3 μm diameter (50 μL=15.3 mg); 3 μm diameter (200 μL=19.4 mg); and 5.17 μm diameter (200 μL=19.8 mg). The above amount of particles were added to 1 mL of 50 mM MES, pH 6, 6.0 M guanidinium thiocyanate containing 25 μg sonicated calf thymus DNA.

The mixtures were incubated for 10 minutes at ambient temperature with occasional mixing. Washing of the solid substrate, elution of the nucleic acid, and quantitation of the eluted nucleic acid was as in Example 1. Experiments were done in triplicate for each microsphere size.

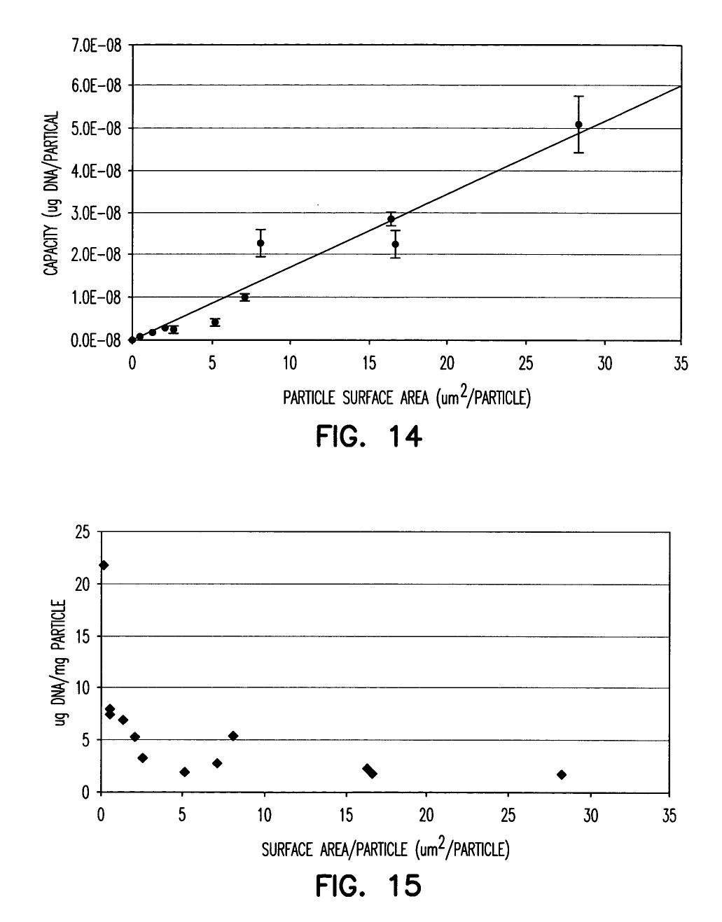

The results are shown in FIG. 14. The results show that DNA binding capacity per particle was approximately linearly proportional to surface area of the microsphere solid substrate. To the extent the results deviated from a linear relationship, this may be due to variability in the actual surface area of the microspheres.

FIG. 15 examines the same data by plotting the amount of DNA recovered per mg of particle as a function of the particle surface area. From this figure, one can calculate the amount (in weight) of particles needed to recover a desired amount of DNA. For recovering a given amount of DNA, the amount of particles required will depend on the surface area of the particles.

The data show that smaller solid substrate microspheres can bind more DNA per unit mass of particle. The amount of DNA bound per mg particle increases sharply as particle surface area decreases below about 3 μm² per particle.

### Example 19

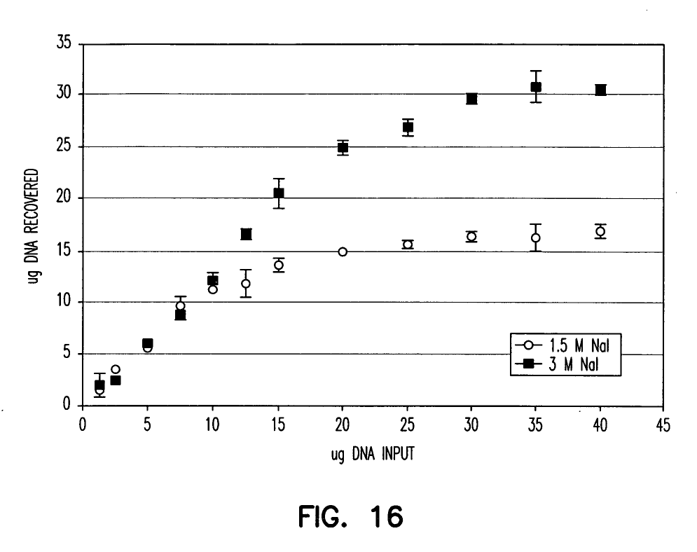

This Example shows that the binding capacity of the solid substrate can be controlled by salt concentration. SIGMA silica (10 mg) was incubated with sheared calf thymus DNA in 1 ml of 50 mM AMP, pH 10, containing either 1.5 M or 3 M NaI, for 10 minutes at room temperature with varying amounts of DNA. Washing of the solid substrate, elution of the nucleic acid, and quantitation of the eluted nucleic acid was as in Example 1. The results are shown in FIG. 16. The results show that the silica solid substrate had saturation kinetics for binding DNA in both salt concentrations. The amount of DNA bound plateaued at about 17 μg in 1.5 M NaI and about 31 μg in 3 M NaI. In addition, the recovery of DNA at subsaturating levels was equivalent at the two salt concentrations, indicating that the particle capacity, and not the affinity, was being altered by the salt concentration.

The invention is described with reference to various specific and preferred embodiments and techniques. However, it should be understood that many variations and modifications may be made while remaining within its scope.

All referenced publications, patents, and patent documents are incorporated by reference, as though individually incorporated by reference.

What is claimed is:

1. A method for binding a predetermined amount of a nucleic acid, the method comprising:
contacting one or more sample solutions comprising the nucleic acid with a multiplicity of solid substrate binding units, wherein the solid substrate binding units bind the nucleic acid, and wherein each binding unit has a predetermined binding capacity for the nucleic acid.

2. The method of claim 1, wherein the binding capacity is determined by the surface area of each solid substrate binding unit, the pH of the one or more sample solutions, and the concentrations of a cation and an anion of a salt in the one or more sample solutions.

3. The method of claim 2, wherein the cation is lithium, sodium, potassium, rubidium, tetramethyl ammonium, tetraethyl ammonium, tetrapropyl ammonium, tetrabutyl ammonium, or a combination thereof.

4. The method of claim 2, wherein the anion is chloride, fluoride, phosphate, sulfate, sulfide, bromide, iodide, perchlorate, thiocyanate, picrate, tannate, tungstate, molybdate, acetate, trichloroacetate, sulfosalicylate, tribromoacetate, thiocyanate, trichloroacetate, nitrate, or combinations thereof.

5. The method of claim 2, wherein the salt is sodium iodide.

6. The method of claim 1, further comprising after the contacting step the step of washing the binding units to remove components other than the nucleic acid.

7. The method of claim 1, wherein the nucleic acid is DNA.

8. The method of claim 2, wherein the solid substrate binding units preferentially bind DNA over RNA.

9. The method of claim 8, wherein the solid substrate binding units substantially bind DNA and substantially do not bind RNA.

10. The method of claim 9, wherein the pH of the one or more sample solutions is at least about 8 and the anion has a molecular radius at least as large as bromide and is at a concentration of at least about 1 M.

11. The method of claim 10, wherein the pH is about 8 to about 12.

12. The method of claim 11, wherein the pH is about 9 to about 12.

13. The method of claim 12, wherein the pH is about 9 to about 11.

14. The method of claim 10, wherein the anion concentration is at least about 2 M.

15. The method of claim 14, wherein the anion concentration is at least about 3 M.

16. The method of claim 10, wherein the anion is a monovalent anion.

17. The method of claim 10, wherein the anion is bromide, iodide, perchlorate, thiocyanate, picrate, tannate, tungstate, molybdate, trichloroacetate, sulfosalicylate, tribromoacetate, or nitrate.

18. The method of claim 10, wherein the cation is sodium.

19. The method of claim 10, wherein the salt is sodium iodide.

20. The method of claim 1, wherein the solid substrate binding units comprise a silica-based material.

21. The method of claim 20, wherein the silica-based material is silica, diatomaceous earth, glass, quartz, or silica gel.

22. The method of claim 1, wherein the solid substrate binding units comprise an anion exchange group.

23. The method of claim 1, wherein the solid substrate binding units comprise zirconium, titanium dioxide, a dye, a hydrophobic interaction group, an oligonucleotide, or an antibody.

24. The method of claim 1, wherein the solid substrate binding units are magnetic.

25. The method of claim 1, wherein the nucleic acid is RNA.

26. The method of claim 25, wherein both RNA and DNA are simultaneously bound to the solid substrate binding units during the contacting.

27. The method of claim 26, further comprising selectively eluting the RNA from each solid substrate binding unit by contacting the binding unit with elution buffer.

28. The method of claim 26, wherein the elution buffer has a pH of at least about 8 and comprises a salt consisting of a cation and an anion, wherein the anion has a molecular radius at least as large as bromide and is at a concentration of at least about 1 M.

29. The method of claim 28, wherein the solid substrate binding units comprise a silica-based material.

30. The method of claim 29, wherein the silica-based material is silica, silicon dioxide, diatomaceous earth, glass, quartz, or silica gel.

31. The method of claim 1, wherein before the contacting step, the sample is purified to remove one or more undesired components.

32. The method of claim 31, wherein the undesired component is protein.

33. The method of claim 31, wherein the nucleic acid is RNA and the undesired component is DNA.

34. The method of claim 1, wherein the nucleic acid is a mixture of DNA and RNA.

35. The method of claim 1, wherein the solid substrate binding units are containers.

36. The method of claim 35, wherein the solid substrate binding units are wells of a multiwell plate.

37. The method of claim 1, wherein the solid substrate binding units are spheres or polyhedrons.

38. The method of claim 1, wherein the solid substrate binding units are microchannels.

39. An apparatus for binding one or more predetermined amounts of a nucleic acid, the apparatus comprising a multiplicity of solid substrate binding units, wherein the solid substrate binding units bind the nucleic acid under appropriate conditions, wherein each binding unit has a substantially identical binding capacity for the nucleic acid as compared to the other binding units, or has a binding capacity that is a predetermined ratio of the binding capacity of another binding unit; and wherein the binding units are operably linked to form an array or are arranged so as to make simultaneous contact with a single sample solution comprising the nucleic acid.

40. The apparatus of claim 39, wherein the solid substrate binding units are non-spherical.

41. The apparatus of claim 39 wherein each solid substrate binding unit has a substantially identical binding capacity for the nucleic acid as compared to the other binding units.

42. The apparatus of claim 39, wherein the solid substrate binding units are arranged so as to make simultaneous contact with a single sample solution comprising the nucleic acid.

43. The apparatus of claim 39, wherein the solid substrate binding units are operably linked to form an array.

44. The apparatus of claim 39, wherein the nucleic acid is DNA.

45. The apparatus of claim 39, wherein the nucleic acid is RNA.

46. The apparatus of claim 39, wherein the nucleic acid is a mixture of DNA and RNA.

47. The apparatus of claim 39, wherein the solid substrate binding units comprise a silica-based material.

48. The apparatus of claim 47, wherein the silica-based material is silica, diatomaceous earth, glass, quartz, or silica gel.

49. The apparatus of claim 39, wherein the solid substrate binding units comprise an anion exchange group.

50. The apparatus of claim 39, wherein the solid substrate binding units comprise zirconium, titanium dioxide, a dye, a hydrophobic interaction group, an oligonucleotide, or an antibody.

51. The apparatus of claim 39, wherein the solid substrate binding units are containers.

52. The apparatus of claim 51, wherein the solid substrate binding units are wells of a multiwell plate.

53. The apparatus of claim 39, wherein the solid substrate binding units are polyhedrons.

54. The apparatus of claim 39, wherein the solid substrate binding units are microchannels.
